(19) United States
(12) Patent Application Publication
(10) Pub. No.: US 2009/0105124 A1
(43) Pub. Date: Apr. 23, 2009

(54) HETEROCYCLIC MODULATORS OF TGR5

(75) Inventors: Nicholas D. Smith, San Diego, CA (US); Joseph E. Payne, Oceanside, CA (US); Timothy Z. Hoffman, San Diego, CA (US); Celine Bonnefous, San Diego, CA (US); Anothony B. Pinkerton, San Diego, CA (US); Dana L. Siegel, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignee: Kalypsys, Inc., San Diego, CA (US)

(21) Appl. No.: 12/193,579

(22) Filed: Aug. 18, 2008

Related U.S. Application Data
(60) Provisional application No. 60/957,522, filed on Aug. 23, 2007.

(51) Int. Cl. A61K 38/28; C07D 475/02; C07D 471/04; A61P 3/10; A61P 19/02; A61P 3/00; A61P 29/00; A61K 31/4985; A61K 31/5025

(52) U.S. Cl. 514/4; 544/258; 544/279; 514/249; 514/264.1

(57) ABSTRACT

Disclosed herein are new heterocyclic compounds and compositions and their application as pharmaceuticals for the treatment of disease. Methods of modulation of TGR5 activity in a human or animal subject are also provided for the treatment diseases mediated by TGR5.

This application claims the benefit of priority of U.S. provisional application No. 60/957,522, filed Aug. 23, 2007, the disclosure of which is hereby incorporated by reference as if written herein in its entirety.

Disclosed herein are new heterocyclic compounds and compositions and their application as pharmaceuticals for the treatment of disease. Methods of modulation of TGR5 activity in a human or animal subject are also provided for the treatment diseases mediated by TGR5.

Obesity is a growing threat to the global health by virtue of its association with a cluster of diseases that include insulin resistance, glucose intolerance, dyslipidemia, and hypertension, collectively known as the metabolic syndrome or syndrome X. It is well documented that patients with metabolic syndrome have a higher risk for coronary heart disease and stroke [Grundy S. M. et al. Circulation 112:e285-e290, 2005]. The treatment of obesity will require complex solutions, including increased public awareness to diminish food portions, improved food choices and increased physical activity. However, epidemiologic studies have shown that treating diabetes/insulin resistance in these patients can reduce the risk of coronary artery disease. Marketed drugs to treat diabetes and insulin resistance include biguanides (such as metformin), peroxisome proliferator activated receptor gamma (PPARγ) agonists (such as rosiglitazone and pioglitazone), sulphonylureas, and most recently GLP-1 mimetics such as Exenatide (Byetta). However, there remains a need for additional agents that can perhaps treat the root cause(s) of metabolic syndrome by treating obesity and diabetes. TGR5 modulators described in this invention might represent such an opportunity.

Bile acids (BA) are amphipathic molecules which are synthesized in the liver from cholesterol and stored in the gall bladder until secretion to the duodenum and intestine to play an important role in the solubilization and absorption of dietary fat and lipid-soluble vitamins. Approx. 99% of BA are absorbed again by passive diffusion and active transport in the terminal ileum and transported back to the liver via the portal vein (enterohepatic circulation). In the liver, BA decrease their own biosynthesis from cholesterol through the activation of the farnesoid X receptor alpha (FXRα) and small heterodimer partner (SHP), leading to the transcriptional repression of cholesterol 7α-hydroxylase, the rate-limiting step of BA biosynthesis from cholesterol.

Recently, two groups independently discovered the GPCR, TGR5 (aka M-BAR) which responds to bile acids [Kawamata Y. et al, J. Biol. Chem., 278:9435-9440, 2003; Maruyama T. et al. Biochem. Biophs. Res. Commun. 298, 714-719, 2002]. TGR5 is a seven transmembrane Gs-coupled GPCR and stimulation by ligand binding causes activation of adenylyl cyclase which leads to the elevation of intracellular cAMP and subsequent activation of downstream signaling pathways. The human receptor shares 86, 90, 82, and 83% amino acid identity to bovine, rabbit, rat, and mouse receptor, respectively. TGR5 is abundantly expressed in the lung, spleen, small intestine, placenta and mononuclear cells (Kawamata Y. et al, J. Biol. Chem., 278:9435-9440, 2003). Bile acids induced receptor internalization, intracellular cAMP production and activation of extracellular signal-regulated kinase in TGR5-expressing HEK293 and CHO cells. In addition, TGR5 was found to be abundantly expressed in monocytes/macrophages from humans and rabbits (Kawamata Y. et al, J. Biol. Chem., 278:9435-9440, 2003), and bile acid treatment suppressed LPS-induced cytokine production in rabbit alveolar macrophages and human THP-1 cells expressing TGR5. These data suggest that bile acids can suppress the macrophage function via activation of TGR5.

Maruyama et al. [Maruyama T. et al. Biochem. Biophs. Res. Commun. 298, 714-719, 2002] showed that TGR5 is expressed in intestinal enteroendocrine cell lines from human (NCI—H716) and murine (STC-1, GLUTag) origin, but not in the intestinal epithelial cells (CaCo-2 and HT-29). Stimulation of TGR5 by BA in NCI—H716 cells stimulated cAMP production. This suggested that bile acids may induce the secretion of glucagon-like peptide-1 (GLP-1) or cholecystokinin (CCK) from the enteroendocrine cells through TGR5 stimulation, since cAMP stimulated the secretion of GLP-1 and CCK from these cells [Reimer R. A. et al. Endocrinology 142, 4522-4528, 2001; Chang C. H. et al. Am. J. Physiol. 271, G516-G523, 1996; Brubaker P. L. et al, Endocrinology 139, 4108-4114, 1998]. This hypothesis was recently confirmed in a publication by Katsuma S. et al. who demonstrated that activation of TGR5 by BA promoted GLP-1 in STC-1 cells [Katsuma S. et al. Biochem. Biophys. Res. Commun. 329, 386-390, 2005]. RNA interference experiments revealed that reduced expression of TGR5 resulted in reduced secretion of GLP-1. GLP-1 has been shown to stimulate insulin release in a glucose dependent manner in humans [Kreymann et al. Lancet 2 (8571) 1300-1304, 1987] and studies in experimental animals demonstrated that this incretin hormone is necessary for normal glucose homeostasis. In addition, GLP-1 can exert several beneficial effects in diabetes and obesity, including 1) increased glucose disposal, 2) suppression in glucose production, 3) reduced gastric emptying, 4) reduction in food intake and 5) weight loss.

Furthermore, recently published data suggested that activation of TGR5 might be beneficial for the treatment of obesity and diabetes. Watanabe et al. (Nature, 439, 484-489, 2006) reported that mice fed high fat diet (HFD) containing 0.5% cholic acid gained less weight than control mice on HFD alone. There was no difference between the two groups in terms of food intake. These effects were independent of FXR-alpha, and instead stem from the binding of bile acids to TGR5 and the subsequent induction of the cAMP-dependent thyroid hormone activating enzyme type 2 (D2) which converts the inactive T3 into the active T4, leading to stimulation of the thyroid hormone receptor and promoting energy expenditure. Mice lacking the D2 gene (D2^{-/-}) were resistant to cholic acid-induced weight loss. In both rodents and humans, the most thermogenically important tissues (the brown adipose and skeletal muscle) are specifically targeted by this mechanism because they co-express D2 and TGR5. The BA-TGR5-cAMP-D2 signaling pathway is therefore a crucial mechanism for fine-tuning energy homeostasis that can be targeted to improve metabolic control.

Taken together, a small molecule TGR5 modulator could be used for the treatment of obesity, diabetes and a wide range of acute and chronic inflammatory diseases.

Recently, certain substituted heterocyclic compounds have been described as agonists of TGR5 for the treatment of metabolic, cardiovascular, and inflammatory diseases (EP01/591120A1, WO04/043468A1, WO04/067008A1, and JP24346059A2).

Novel compounds and pharmaceutical compositions, certain of which have been found to modulate TGR5 have been discovered, together with methods of synthesizing and using the compounds including methods for the treatment of TGR5-mediated diseases in a patient by administering the compounds.

In certain embodiments of the present invention, compounds have structural Formula I:

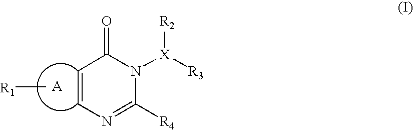

or a salt, ester, or prodrug thereof, wherein:

A is a 5 or 6-membered monocyclic heterocycloalkyl or cycloalkyl;

X is selected from the group consisting of CH and N;

R₁ is selected from the group consisting of hydrogen, halogen, amino, cyano, nitro, hydroxy, alkoxy, alkyl, acyl, alkenyl, alkynyl, heteroalkyl, carboxyl, alkylthio, alkylsulfonyl, sulfonamido, aryl, arylalkyl, cycloalkyl, cycloalkylalkyl, haloalkyl, perhaloalkyl, perhaloalkoxy, heteroaryl, heteroarylalkyl, heterocycloalkyl, and heterocycloalkylalkyl, any of which may be optionally substituted;

R₂ and R₃ are independently selected from the group consisting of hydrogen, hydroxy, alkoxy, aryloxy, alkyl, acyl, alkenyl, alkynyl, heteroalkyl, carboxyl, alkylthio, alkylsulfonyl, sulfonamido, aryl, arylalkyl, cycloalkyl, cycloalkylalkyl, haloalkyl, perhaloalkyl, perhaloalkoxy, heteroaryl, heteroarylalkyl, heterocycloalkyl, and heterocycloalkylalkyl, any of which may be optionally substituted; and

R₄ is selected from the group consisting of hydrogen, halogen, amino, cyano, nitro, hydroxy, alkoxy, alkyl, acyl, alkenyl, alkynyl, heteroalkyl, carboxyl, alkylthio, alkylsulfonyl, sulfonamido, aryl, arylalkyl, cycloalkyl, cycloalkylalkyl, haloalkyl, perhaloalkyl, perhaloalkoxy, heteroaryl, heteroarylalkyl, heterocycloalkyl, and heterocycloalkylalkyl, arylthio, and heterocycloalkylthio, any of which may be optionally substituted.

Certain compounds disclosed herein may possess useful TGR5 modulating activity, and may be used in the treatment or prophylaxis of a disease or condition in which TGR5 plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for modulating TGR5. Other embodiments provide methods for treating a TGR5-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the modulation of TGR5 activity.

In certain embodiments, the compounds have structural Formula II:

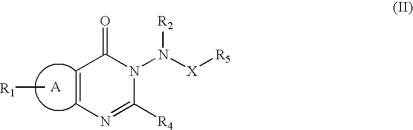

or a salt, ester, or prodrug thereof, wherein:

A is a 5 or 6-membered monocyclic heterocycloalkyl or heteroaryl;

X is selected from the group consisting of C(R₆)(R₇) and C(R₆)(R₇)C(R₈)(R₉).
R₁ is selected from the group consisting of hydrogen, halogen, amino, cyano, nitro, hydroxy, alkoxy, alkyl, acyl, alkenyl, alkynyl, heteroalkyl, carboxyl, alkylthio, alkylsulfonyl, sulfonamido, aryl, arylalkyl, cycloalkyl, cycloalkylalkyl, haloalkyl, perhaloalkyl, perhaloalkoxy, heteroaryl, heteroarylalkyl, heterocycloalkyl, and heterocycloalkylalkyl, any of which may be optionally substituted;

R₂ is selected from the group consisting of hydrogen, alkyl, acyl, alkenyl, alkynyl, heteroalkyl, aryl, arylalkyl, cycloalkyl, cycloalkylalkyl, haloalkyl, perhaloalkyl, heteroaryl, heteroarylalkyl, heterocycloalkyl, and heterocycloalkylalkyl, any of which may be optionally substituted, or R₂ and R₆, taken together, form a bond;

R₄ and R₅ are independently selected from the group consisting of aryl, cycloalkyl, heteroaryl, and heterocycloalkyl, any of which may be optionally substituted; and

R₆, R₇, R₈, and R₉ are independently selected from the group consisting of hydrogen and optionally substituted lower alkyl, or R₆ and R₇ or R₈ and R₉, taken together, are oxo or saturated C₃-C₆ cycloalkyl.

In further embodiments, X is selected from the group consisting of CH₂ and CH₂CH₂; and R₂ is selected from the group consisting of hydrogen and lower alkyl.

In yet further embodiments, A is a 5 or 6-membered monocyclic heteroaryl.

In yet further embodiments, R₄ and R₅ are independently selected from the group consisting of phenyl, napthyl, monocyclic heteroaryl, and bicyclic heteroaryl, any of which may be optionally substituted with one or more substituents selected from the group consisting of hydrogen, halogen, hydroxyl, nitro, cyano, amino, lower alkyl, lower alkoxy, and lower perhaloalkyl.

In further embodiments, the compounds have a structural formula selected from the group consisting of Formula III and Formula IV:

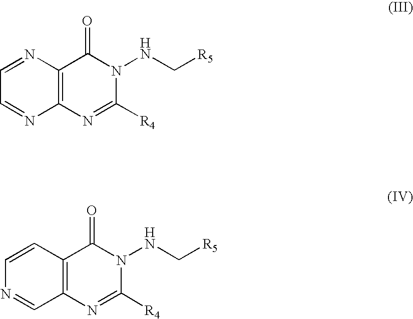

or a salt, ester, or prodrug thereof, wherein R₄ and R₅ are independently selected from the group consisting of phenyl, napthyl, monocyclic heteroaryl, and bicyclic heteroaryl, any of which may be optionally substituted with one or more substituents selected from the group consisting of hydrogen, halogen, hydroxyl, nitro, cyano, amino, lower alkyl, lower alkoxy, and lower perhaloalkyl.

In yet further embodiments, R₄ and R₅ are each phenyl, which may be optionally substituted with one or more substituents selected from the group consisting of hydrogen, halogen, hydroxyl, nitro, cyano, amino, lower alkyl, lower alkoxy, and lower perhaloalkyl.

In yet further embodiments, R₅ is 4-halophenyl.

In yet further embodiments, R₄ is selected from the group consisting of 5-chloro-pyridin-3-yl, 5-methyl-pyridin-3-yl, 4-fluorophenyl, 4-amino-3-methylphenyl, 4-hydroxy-3-methylphenyl, and 4-hydroxy-3-methoxyphenyl.

In further embodiments, the compound is selected from the group consisting of Examples 1 to 51.

In other embodiments, said disease is a metabolic disease.

In further embodiments, said disease is selected from the group consisting of inadequate glucose tolerance, insulin resistance, type I diabetes, and type II diabetes.

In other embodiments, said disease is associated with perturbed bile acid metabolism.

In other embodiments, said disease is an inflammatory disease.

In further embodiments, said disease is selected from the group consisting of rheumatoid arthritis, ulcerative colitis, and inflammatory bowel disease.

In other embodiments, said disease is obesity.

In further embodiments, said method achieves an effect selected from the group consisting of decreasing body weight and controlling weight gain.

In yet further embodiments, said method further comprises the administration of another therapeutic agent.

In yet further embodiments, said agent is selected from the group consisting of insulin, metformin, Glipizide, glyburide, Amaryl, gliclazide, meglitinides, nateglinide, repaglinide, pramlintide, PTP-112, SB-517955, SB-4195052, SB-216763, NN-57-05441, NN-57-05445, GW-0791, AGN-¹⁹4²⁰4, T-1095, BAY R3401, acarbose, miglitol, voglibose, Exendin-4, DPP728, LAF237, vildagliptin, BMS477118, PT-100, GSK-823093, PSN-9301, T-6666, SYR-322, SYR-619, Liraglutide, CJC-1134-PC, naliglutide, MK-0431, saxagliptin, GSK23A, pioglitazone, rosiglitazone, AVE2268, GW869682, GSK189075, APD668, PSN-119-1, PSN-821, rosuvastatin, atrovastatin, simvastatin, lovastatin, pravastatin, fluvastatin, cerivastatin, rosuvastatin, pitavastatin, fenofibrate, benzafibrate, clofibrate, gemfibrozil, Ezetimibe, eflucimibe, CP-529414, CETi-1, JTT-705, cholestyramine, colestipol, niacin, implitapide, (R)-1-{4-[5-methyl-2-(4-trifluoromethyl-phenyl)-oxazol-4-ylmethoxy]-benzenesulfonyl}2,3-dihydro-1H-indole-2-carboxylic acid, and GI-262570.

In yet further embodiments, said agent is selected from the group consisting of betamethasone dipropionate, betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocaine, EMLA Cream, guaifenesin, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin, clonidine, codeine, loperamide, tramadol, morphine, fentanyl, oxycodone, hydrocodone, levorphanol, butorphanol, menthol, oil of wintergreen, camphor, eucalyptus oil, turpentine oil, acetaminophen, infliximab, etanerecept, infliximab, and capsaicin.

In yet further embodiments, said agent is selected from the group consisting of sibutramine, bromocriptine, Orlistat, rimonabant, Axokine, and bupropion.

In other embodiments, said method achieves an effect in a patient comprising the administration of a therapeutically effective amount of a compound as recited in Claim 1 to a patient, wherein the effect is selected from the group consisting of improving glucose tolerance, decreasing insulin resistance, decreasing body weight, controlling weight gain, modulation of type I diabetes, modulation of type II diabetes, modulation of perturbed bile acid metabolism, modulation of rheumatoid arthritis, modulation of ulcerative colitis, and modulation of inflammatory bowel disease.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon group having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “xaralkanoyl” or “aroyl,” as used herein, alone or in combination, refers to an acyl group derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent group C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“TGR5 modulator” is used herein to refer to a compound that exhibits an EC₅₀ with respect to TGR5 activity of no more than about 100 μM and more typically not more than about 50 μM, as measured in the cAMP production assay and glucagon-like peptide-1 (GLP-1) secretion assays described generally hereinbelow. “EC₅₀” is that concentration of inhibitor which activates the activity of an enzyme (e.g., TGR5) to half-maximal level. Certain compounds disclosed herein have been discovered to exhibit modulatory activity against TGR5. In certain embodiments, compounds will exhibit an EC₅₀ with respect to TGR5 of no more than about 10 μM; in further embodiments, compounds will exhibit an EC₅₀ with respect to TGR5 of no more than about 5 μM; in yet further embodiments, compounds will exhibit an EC₅₀ with respect to TGR5 of not more than about 1 μM; in yet further embodiments, compounds will exhibit an EC₅₀ with respect to TGR5 of not more than about 200 nM, as measured in the TGR5 assay described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

In certain embodiments, the salts may include hydrochloride and trifluoroacetic acid salts of compounds disclosed herein. A salt of a compound can be made by reacting the appropriate compound in the form of the free base with the appropriate acid.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention with agents found in the following pharmacotherapeutic classifications as indicated below. These lists should not be construed to be closed, but should instead serve as illustrative examples common to the relevant therapeutic area at present. Moreover, combination regimens may include a variety of routes of administration and should include oral, intravenous, intraocular, subcutaneous, dermal, and inhaled topical.

For the treatment of metabolic disorders, compounds according to the present invention may be administered with an agent selected from the group comprising: insulin, insulin derivatives and mimetics, insulin secretagogues, insulin sensitizers, biguanide agents, alpha-glucosidase inhibitors, insulinotropic sulfonylurea receptor ligands, protein tyrosine phosphatase-1B (PTP-1B) inhibitors, GSK3 (glycogen synthase kinase-3) inhibitors, GLP-1 (glucagon like peptide-1), GLP-1 analogs, DPPIV (dipeptidyl peptidase IV) inhibitors, RXR ligands sodium-dependent glucose co-transporter inhibitors, glycogen phosphorylase A inhibitors, an AGE breaker, PPAR modulators, and non-glitazone type PPARδ agonist.

For the treatment of metabolic disorders, compounds according to the present invention may be administered with an agent selected from the group comprising: insulin, metformin, Glipizide, glyburide, Amaryl, gliclazide, meglitinides, nateglinide, repaglinide, pramlintide, PTP-112, SB-517955, SB-4195052, SB-216763, NN-57-05441, NN-57-05445, GW-0791, AGN-¹⁹4²⁰4, T-1095, BAY R3401, acarbose, miglitol, voglibose, Exendin-4, DPP728, LAF237, vildagliptin, BMS477118, PT-100, GSK-823093, PSN-9301, T-6666, SYR-322, SYR-619, Liraglutide, CJC-1134-PC, naliglutide, MK-0431, saxagliptin, GSK23A, pioglitazone, rosiglitazone, AVE2268, GW869682, GSK189075, APD668, PSN-119-1, PSN-821, rosuvastatin, atorvastatin, simvastatin, lovastatin, pravastatin, fluvastatin, cerivastatin, rosuvastatin, pitavastatin, fenofibrate, benzafibrate, clofibrate, gemfibrozil, Ezetimibe, eflucimibe, CP-529414, CETi-1, JTT-705, cholestyramine, colestipol, niacin, implitapide, (R)-1-{4-[5-methyl-2-(4-trifluoromethyl-phenyl)-oxazol-4-ylmethoxy]-benzenesulfonyl}2,3-dihydro-1H-indole-2-carboxylic acid, and GI-262570.

For the treatment of inflammatory diseases, compounds according to the present invention may be administered with an agent selected from the group comprising: corticosteroids, non-steroidal anti-inflammatories, muscle relaxants and combinations thereof with other agents, anaesthetics and combinations thereof with other agents, expectorants and combinations thereof with other agents, antidepressants, anticonvulsants and combinations thereof, antihypertensives, opioids, topical cannabinoids, and other agents, such as capsaicin.

For the treatment of inflammatory diseases, compounds according to the present invention may be administered with an agent selected from the group comprising: betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocaine, EMLA Cream (Eutectic Mixture of Local Anesthetics (lidocaine 2.5% and prilocaine 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab; n) nitric oxide synthase inhibitors, particularly inhibitors of inducible nitric oxide synthase; and other agents, such as capsaicin.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating TGR5-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of TGR5-mediated disorders.

Specific diseases to be treated by the compounds, compositions, and methods disclosed herein include: diabetes (type I and type II) and conditions associated with diabetic diseases which include, but are not limited to, hyperglycemia, hyperlipidemia, hyperinsulinemia, insulin resistance, inadequate glucose tolerance, impaired glucose metabolism, diabetic nephropathy, glomerulosclerosis, diabetic neuropathy, erectile dysfunction, macular degeneration, diabetic retinopathy, chronic microvascular complications, peripheral vascular disease, cataracts, stroke, foot ulcerations, renal failure, kidney disease, ketosis, metabolic acidosis, and related disorders, obesity, myocardial infarction, angina pectoris, coronary artery disease, atherosclerosis, cardiac hypertrophy, allergic diseases, fatty liver disease, nonalcoholic steatohepatitis, liver fibrosis, kidney fibrosis, anorexia nervosa, bulimia nervosa, autoimmune diseases, inflammatory diseases including rheumatoid arthritis, asthma, chronic obstructive pulmonary disease (COPD), psoriasis, ulcerative colitis, proliferative disorders, infectious diseases, angiogenic disorders, reperfusion/ischemia in stroke, vascular hyperplasia, organ hypoxia, cardiac hypertrophy, thrombin-induced platelet aggregation, and conditions associated with prostaglandin endoperoxidase synthetase-2 (COX-2).

In certain embodiments, the disease is obesity and the effects to be achieved in a human or animal patient include decreasing body weight and controlling weight gain.

In addition, topical application of TGR5 agonists might be useful for the treatment of cellulite and other cosmetic conditions which are characterized by subcutaneous fat accumulation. This is due to recent evidence showing that TGR5 agonists increase energy expenditure and fat burning in experimental models (Watanabe et al. Nature, 439:484-489).

In certain embodiments, the disease is associated with perturbed bile acid metabolism, including, but not limited to gall bladder stones, cholecystitis, cholangitis, choledocholithiasis, jaundice, and obstetric cholestasis and the itch associated with it.

Metabolic diseases other than Type 1 and Type 2 diabetes which may be treated or prevented include, without limitation, metabolic syndrome and insulin resistance. In addition, the compounds disclosed herein can be used to treat insulin resistance and other metabolic disorders such as atherosclerosis that are typically associated with an exaggerated inflammatory signaling.

In certain embodiments, the disease is a hyperproliferative condition of the human or animal body, including, but not limited to restenosis, inflammation, immune disorders, cardiac hypertrophy, atherosclerosis, pain, migraine, angiogenesis-related conditions or disorders, proliferation induced after medical conditions, including but not limited to surgery, angioplasty, or other conditions.

The compounds disclosed herein may be useful as anti-inflammatory agents with the additional benefit of having significantly less harmful side effects. The compositions may be used to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compositions may also be used in the treatment of pulmonary inflammation, such as that associated with viral infections and cystic fibrosis. In certain embodiments, the particular inflammatory disease is rheumatoid arthritis.

Further inflammatory diseases which may be prevented or treated include, without limitation: asthma, allergies, respiratory distress syndrome or acute or chronic pancreatitis. Furthermore, respiratory system diseases may be prevented or treated including but not limited to chronic obstructive pulmonary disease, pulmonary fibrosis, ulcerative colitis, inflammatory bowel disease, Crohn's disease, peptic ulceration, gastritis, psoriasis, and skin inflammation.

In certain embodiments, the disease to be treated by the methods provided herein may be an opthalmologic disorder. Opthalmologic diseases and other diseases in which angiogenesis plays a role in pathogenesis, may be treated or prevented and include, without limitation, dry eye (including Sjögren's syndrome), macular degeneration, closed and wide angle glaucoma, retinal ganglion degeneration, ocular ischemia, retinitis, retinopathies, uveitis, ocular photophobia, and of inflammation and pain associated with acute injury to the eye tissue. In certain embodiments, the opthalmologic disease to be treated is glaucomatous retinopathy and/or diabetic retinopathy. In certain embodiments, the opthalmologic condition to be treated is post-operative inflammation or pain as from ophthalmic surgery such as cataract surgery and refractive surgery.

In certain embodiments, the disease to be treated by the methods provided herein may be an autoimmune disease. Autoimmune diseases which may be prevented or treated include, but are not limited to: rheumatoid arthritis, inflammatory bowel disease, inflammatory pain, ulcerative colitis, Crohn's disease, periodontal disease, temporomandibular joint disease, multiple sclerosis, diabetes, glomerulonephritis, systemic lupus erythematosus, scleroderma, chronic thyroiditis, Grave's disease, hemolytic anemia, autoimmune gastritis, autoimmune neutropenia, thrombocytopenia, chronic active hepatitis, myasthenia gravis, atopic dermatitis, graft vs. host disease, and psoriasis. Inflammatory diseases which may be prevented or treated include, but are not limited to: asthma, allergies, respiratory distress syndrome or acute or chronic pancreatitis. In certain embodiments, the particular autoimmune disease is rheumatoid arthritis.

The compounds provided herein are also useful in treating tissue damage in such diseases as vascular diseases, migraine headaches, periarteritis nodosa, thyroiditis, aplastic anemia, Hodgkin's disease, sclerodoma, rheumatic fever, neuromuscular junction disease including myasthenia gravis, white matter disease including multiple sclerosis, sarcoidosis, nephritis, nephrotic syndrome, Behcet's syndrome, polymyositis, gingivitis, periodontis, hypersensitivity, swelling occurring after injury, ischemias including myocardial ischemia, cardiovascular ischemia, and ischemia secondary to cardiac arrest, and the like. These compounds can also be used to treat allergic rhinitis, respiratory distress syndrome, endotoxic shock syndrome, and atherosclerosis.

In certain embodiments, the disease to be treated by the methods of the present invention may be a cardiovascular condition. In certain embodiments, said cardiovascular condition is selected from the group consisting of atherosclerosis, cardiac hypertrophy, idiopathic cardiomyopathies, heart failure, angiogenesis-related conditions or disorders, and proliferation induced after medical conditions, including, but not limited to restenosis resulting from surgery and angioplasty.

In certain embodiments, the disease to be prevented or treated by the methods of the present invention may be autism. Recent data have shown that TGR5 agonists increase the expression and the activity of the enzyme iodothyronine deiodinase type 2 (D2) (Watanabe et al. Nature, 439:484-489). D2 converts inactive thyroxine (T4) into active 3,5,3′-tri-iodothyronine (T3). Recent data have also shown that inhibition of D2 in fetal brain causes a reduction of T3 levels and results in permanent alterations of cerebral cortical architecture reminiscent of these observed in brains of patients with autism. Therefore, a TGR5 agonist (or antagonist) might be useful for the prevention or treatment of autism.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein in their entireties. Where any inconsistencies arise, material literally disclosed herein controls.

### General Synthetic Methods for Preparing Compounds

The following schemes can be used to practice the present invention.

Certain compounds as disclosed herein can be synthesized using the following general synthetic procedure set forth in Scheme I.

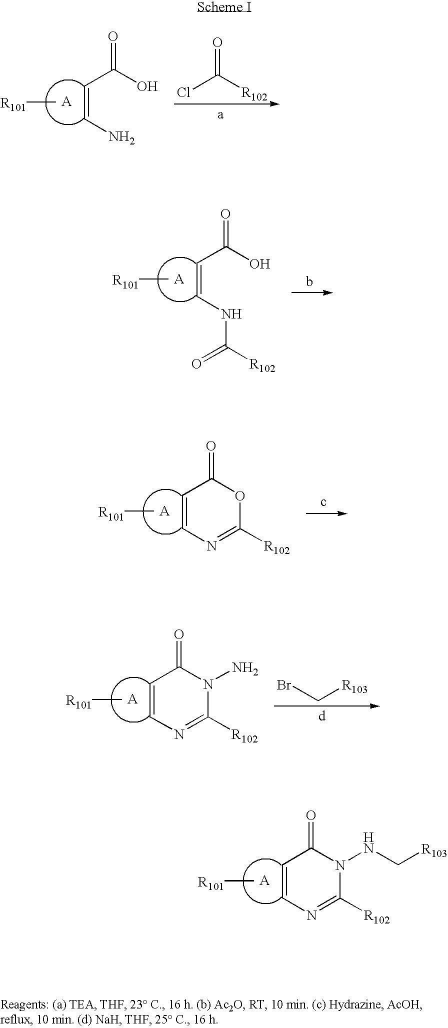

Certain compounds as disclosed herein can be synthesized using the following general synthetic procedure set forth in Scheme II.

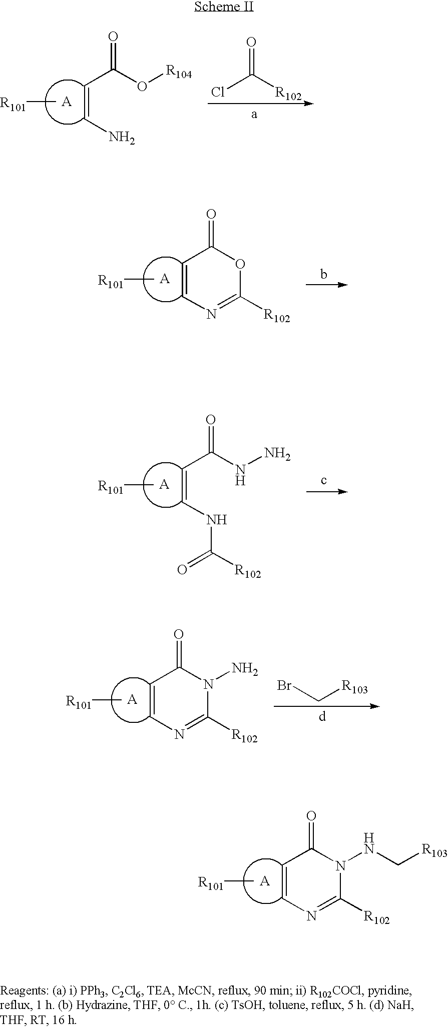

Certain compounds as disclosed herein can be synthesized using the following general synthetic procedure set forth in Scheme III.

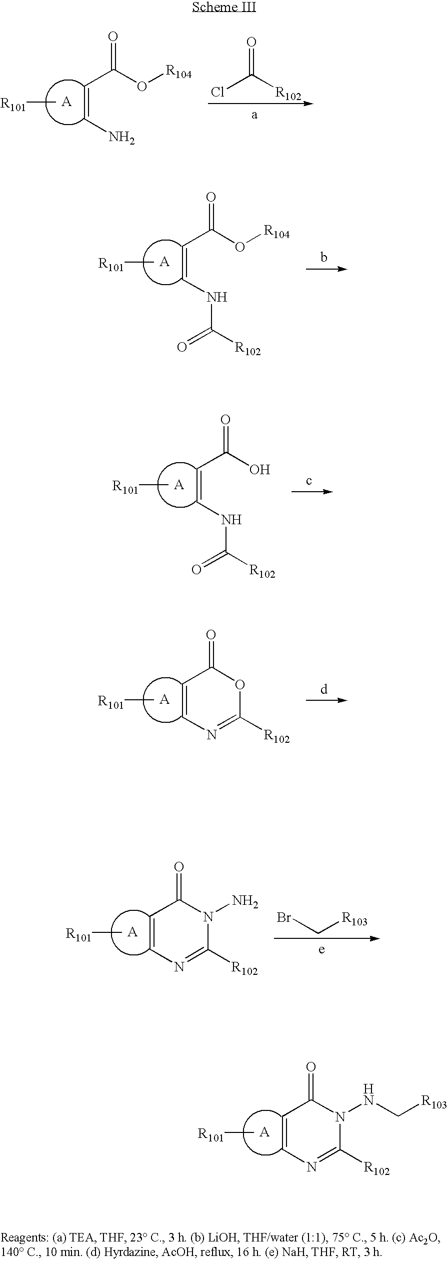

Certain compounds as disclosed herein can be synthesized using the following general synthetic procedure set forth in Scheme IV.

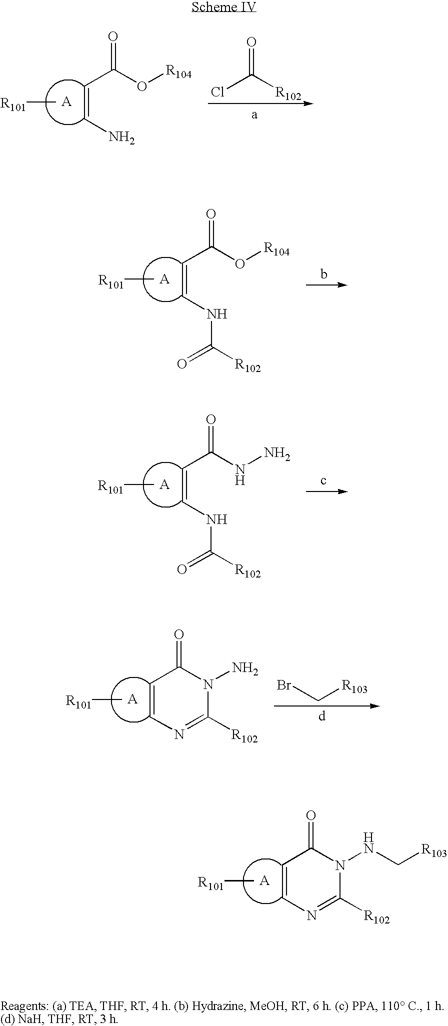

Certain compounds as disclosed herein can be synthesized using the following general synthetic procedure set forth in Scheme V.

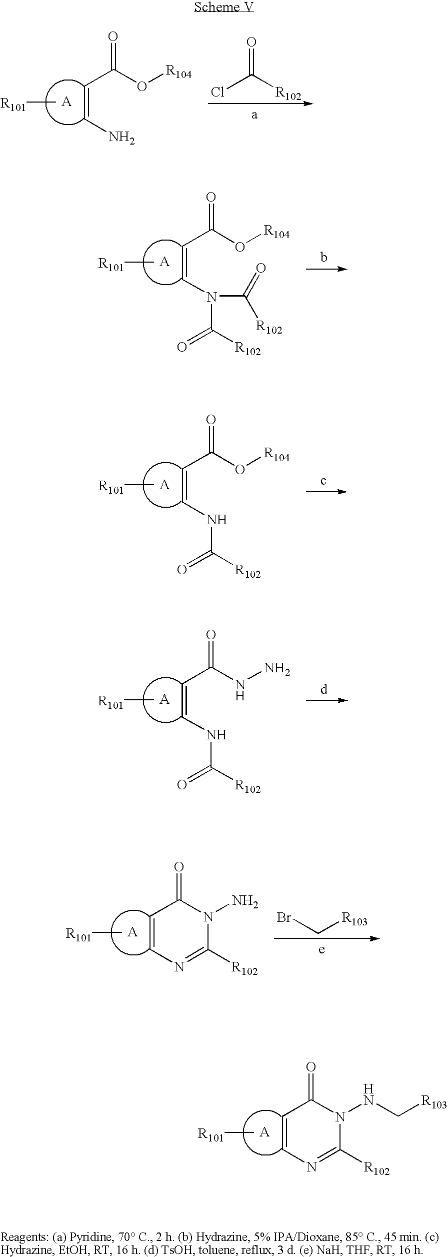

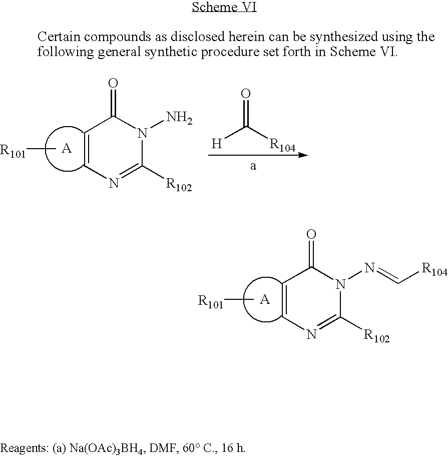

The invention is further illustrated by the following examples.

### EXAMPLE 1

### 3-{[(4-Chloro-2-fluorophenyl)methyl]amino}-2-(5-chloropyridin-3-yl)-3H,4H-pyrido[2,3-d]pyrimidin-4-one

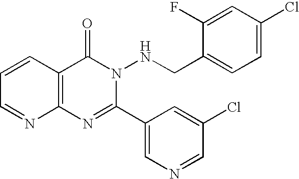

### Step 1: 2-(5-Chloronicotinamido)nicotinic Acid

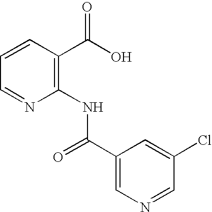

To a solution of 2-aminonicotinic acid (4.4 g) in THF (80 mL), was added TEA (9.7 g), a catalytic amount of DMF (0.080 mL), and a solution of 5-chloronicotinoyl chloride (5.6 g, for the synthesis of 5-chloronicotinoyl chloride, see McElhinney, R. S., et. al.; J. Med. Chem.; 41; 26; 1998; 5625-5271) in THF (70 mL) was then added dropwise, with stirring, at 0° C. The reaction mixture is allowed to warm to RT. After 16 h at RT, the mixture was concentrated. Purification by silica gel chromatography (1% to 20% methanol/DCM) gave 2-(5-chloronicotinamido)nicotinic acid (1.1 g, 11%) as a light yellow solid. LCMS: 278 (M+H)⁺.

### Step 2: 2-(5-Chloropyridin-3-yl)-4H-pyrido[2,3-d][1,3]oxazin-4-one

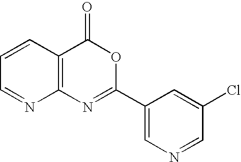

To 2-(5-chloronicotinamido)nicotinic acid (1.1 g) was added acetic anhydride (50 mL). After 10 minutes at RT, the solution is concentrated. Purification by silica gel chromatography (1% methanol/DCM) gave 2-(5-chloropyridin-3-yl)-4H-pyrido[2,3-d][1,3]oxazin-4-one (1 g, 98%). LCMS: 260 (M+H)⁺.

### Step 3: 3-Amino-2-(5-chloropyridin-3-yl)pyrido[2,3-d]pyrimidin-4(3H)-one

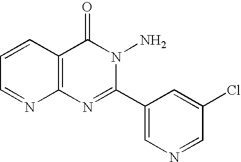

To a solution of 2-(5-chloropyridin-3-yl)-4H-pyrido[2,3-d][1,3]oxazin-4-one (1 g) in acetic acid (50 mL), was added hydrazine (1 g). After 10 minutes at reflux, the reaction mixture was quenched with ice water (40 mL) and filtered. The filter cake was washed with 5% methanol:DCM (3×30 mL) to give 3-amino-2-(5-chloropyridin-3-yl)pyrido[2,3-d]pyrimidin-4(3H)-one (0.42 g, 38%) as a light yellow solid. ¹H NMR (300 MHz, CDCl₃) δ 9.04 (m, 1H), 8.97 (m, 1H), 8.81 (m, 1H), 8.63 (m, 1H), 8.41 (m, 1H), 7.65 (m, 1H), 5.76 (s, 2H). LCMS: 274 (M+H)⁺.

### Step 4: 3-{[(4-Chloro-2-fluorophenyl)methyl]amino}-2-(5-chloropyridin-3-yl)-3H,4H-pyrido[2,3-d]pyrimidin-4-one

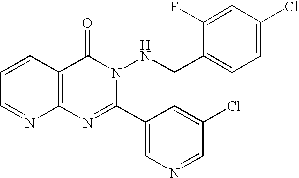

To a slurry of 3-amino-2-(5-chloropyridin-3-yl)pyrido[2,3-d]pyrimidin-4(3H)-one (60 mg) in THF (3 mL), was added sodium hydride (11 mg, 60%) and 1-(bromomethyl)-4-chloro-2-fluorobenzene (40 mg). After 16 h at RT, the reaction mixture was quenched with methanol (1 mL) and concentrated to residue. Purification by silica gel chromatography (50% to 100% ethyl acetate/hexane as the eluting solvent) gave 3-{[(4-chloro-2-fluorophenyl)methyl]amino}-2-(5-chloropyridin-3-yl)-3H,4H-pyrido[2,3-d]pyrimidin-4-one (18 mg, 20%) as a white solid. ¹H NMR (400 MHz, CDCl₃) δ 9.07 (dd, 1H), 8.94 (d, 1H), 8.67 (dd, 1H), 8.62 (d, 1H), 8.01 (t, 1H), 7.54 (m, 1H), 6.94-6.87 (m, 2H), 6.80 (t, 1H), 6.16 (t, 1H), 3.98 (d, 2H). LCMS: 416 (M+H)⁺.

### EXAMPLE 2

### 3-{[(4-Chlorophenyl)methyl]amino}-2-(2-fluorophenyl)-3H,4H-pyrido[2,3-d]pyrimidin-4-one

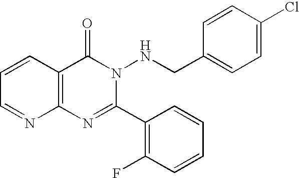

3-{[(4-Chlorophenyl)methyl]amino}-2-(2-fluorophenyl)-3H,4H-pyrido[2,3-d]pyrimidin-4-one was synthesized as described in Example 1 using 2-fluorobenzoyl chloride in step 1, and 1-(bromomethyl)-4-chlorobenzene in step 4. ¹H NMR (400 MHz, CDCl₃) δ 9.05 (dd, 1H), 8.68 (dd, 1H), 7.53-7.49 (m, 2H), 7.41 (td, 1H), 7.26-7.10 (m, 4H), 6.75 (dd, 2H), 5.80 (t, 1H), 3.90 (bs, 2H). LCMS: 378 (M−2H)⁺.

### EXAMPLE 3

### 3-{[(4-Chlorophenyl)methyl]amino}-2-(3-methoxyphenyl)-3H,4H-pyrido[2,3-d]pyrimidin-4-one

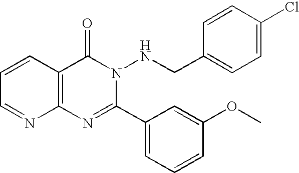

3-{[(4-Chlorophenyl)methyl]amino}-2-(3-methoxyphenyl)-3H,4H-pyrido[2,3-d]pyrimidin-4-one was synthesized as described in Example 1 using 3-methoxybenzoyl chloride in step 1, and 1-(bromomethyl)-4-chlorobenzene in step 4. ¹H NMR (400 MHz, CDCl₃) δ 8.97 (dd, 1H), 8.58 (d, 1H), 7.46 (d, 1H), 7.41 (t, 1H), 7.34-7.28 (m, 2H), 7.08 (d, 2H), 7.00 (d, 1H), 6.83 (d, 2H), 6.00 (t, 1H), 3.79 (s, 3H), 3.75 (bs, 2H). LCMS: 393 (M+H)⁺.

### EXAMPLE 4

### 3-{[(4-Bromophenyl)methyl]amino}-2-(4-fluorophenyl)-3H,4H-pyrido[2,3-d]pyrimidin-4-one

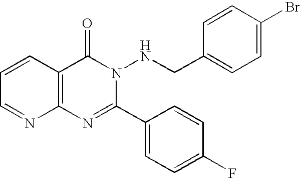

3-{[(4-Bromophenyl)methyl]amino}-2-(4-fluorophenyl)-3H,4H-pyrido[2,3-d]pyrimidin-4-one was synthesized as described in Example 1 using 4-fluorobenzoyl chloride in step 1, and 1-bromo-4-(bromomethyl)benzene in step 4. ¹H NMR (400 MHz, DMSO-d₆) δ 8.99 (m, 1H), 8.58 (d, 1H), 7.71-7.57 (m, 3H), 7.30-7.23 (m, 4H), 6.82-6.78 (m, 3H), 3.89 (m, 2H). LCMS: 427 (M+H)⁺.

### EXAMPLE 5

### 3-{[(4-Bromophenyl)methyl]amino}-2-(4-methoxyphenyl)-3H,4H-pyrido[2,3-d]pyrimidin-4-one

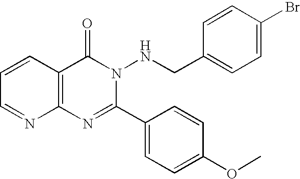

3-{[(4-Bromophenyl)methyl]amino}-2-(4-methoxyphenyl)-3H,4H-pyrido[2,3-d]pyrimidin-4-one was synthesized as described in Example 1 using and 4-methoxybenzoyl chloride in step 1, and 1-bromo-4-(bromomethyl)benzene in step 4. ¹H NMR (400 MHz, DMSO-d₆) δ 8.97-8.95 (m, 1H), 8.54 (dd, 1H), 7.71-7.68 (m, 2H), 7.54 (dd, 1H), 7.30-7.28 (m, 2H), 6.99-6.97 (m, 2H), 6.85-6.83 (m, 3H), 3.87 (m, 2H), 3.84 (s, 3H). LCMS: 439 (M+H)⁺.

### EXAMPLE 6

### 3-{[(4-Bromophenyl)methyl]amino}-2-(4-hydroxyphenyl)-3H,4H-pyrido[2,3-d]pyrimidin-4-one

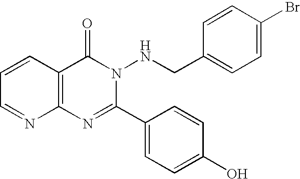

### Step 1: 3-{[(4-Bromophenyl)methyl]amino}-2-(4-methoxyphenyl)-3H,4H-pyrido[2,3-d]pyrimidin-4-one

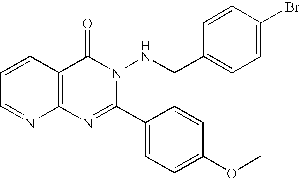

3-{[(4-Bromophenyl)methyl]amino}-2-(4-methoxyphenyl)-3H,4H-pyrido[2,3-d]pyrimidin-4-one was synthesized as described in EXAMPLE 1 using 4-methoxybenzoyl chloride in step 1, and 1-bromo-4-(bromomethyl)benzene in step 4. ¹H NMR (400 MHz, DMSO-d₆) δ 8.97-8.95 (m, 1H), 8.54 (dd, 1H), 7.71-7.68 (m, 2H), 7.54 (dd, 1H), 7.30-7.28 (m, 2H), 6.99-6.97 (m, 2H), 6.85-6.83 (m, 3H), 3.87 (m, 2H), 3.84 (s, 3H). LCMS: 439 (M+H)⁺.

### Step 2: 3-{[(4-Bromophenyl)methyl]amino}-2-(4-hydroxyphenyl)-3H,4H-pyrido[2,3-d]pyrimidin-4-one

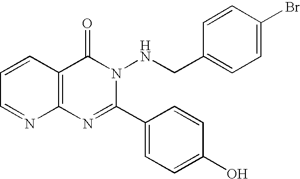

Lithium iodide (167 mg) was added to a solution of 3-{[(4-bromophenyl)methyl]amino}-2-(4-methoxyphenyl)-3H,4H-pyrido[2,3-d]pyrimidin-4-one (109 mg) in collidine (2.5 mL) and heated to 168° C. for 16 h. The reaction mixture was diluted with ethyl acetate and the crude precipitate was filtered. The crude material was taken up in DMSO and purified by preparative HPLC (gradient: 10% to 90% acetonitrile:water) to give 3-{[(4-bromophenyl)methyl]amino}-2-(4-hydroxyphenyl)-3H,4H-pyrido[2,3-d]pyrimidin-4-one as a yellow solid. ¹H NMR (400 MHz, DMSO-d₆) δ 9.98 (s, 1H), 8.96-8.94 (m, 1H), 8.55-8.52 (m, 1H), 7.63 (d, 2H), 7.52 (dd, 1H), 7.30 (d, 2H), 6.87 (d, 2H), 6.82-6.79 (m, 3H), 3.86 (d, 2H). LCMS: 425 (M+H)⁺.

### EXAMPLE 7

### 3-{[(4-Chlorophenyl)methyl]amino}-2-(pyridin-3-yl)-3H,4H-pyrido[2,3-d]pyrimidin-4-one

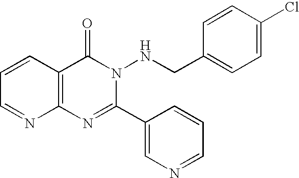

3-{[(4-Chlorophenyl)methyl]amino}-2-(pyridin-3-yl)-3H,4H-pyrido[2,3-d]pyrimidin-4-one was synthesized as described in EXAMPLE 1 using nicotinoyl chloride in step 1, and 1-(bromomethyl)-4-chlorobenzene in step 4. ¹H NMR (400 MHz, CD₃OD, TFA salt) δ 9.03 (s, 1H), 8.82 (m, 2H), 8.56 (m, 1H), 7.87 (m, 1H), 7.70 (m, 1H), 7.31 (m, 1H), 7.07 (d, 2H), 6.81 (d, 2H), 3.98 (bs, 2H). LCMS: 364 (M+H)⁺.

### EXAMPLE 8

### 3-{[(4-Chlorophenyl)methyl]amino}-2-(5-chloropyridin-3-yl)-3H,4H-pyrido[2,3-d]pyrimidin-4-one

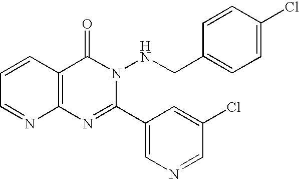

3-{[(4-Chlorophenyl)methyl]amino}-2-(5-chloropyridin-3-yl)-3H,4H-pyrido[2,3-d]pyrimidin-4-one was synthesized as described in EXAMPLE 1 using 1-(bromomethyl)-4-chlorobenzene in step 4. ¹H NMR (400 MHz, DMSO-d₆, HCl salt) δ 9.03 (d, 1H), 8.67 (m, 2H), 7.87 (s, 1H), 7.67 (dd, 1H), 7.37 (s, 1H), 7.14 (d, 2H), 6.79 (d, 2H), 3.95 (bs, 2H). LCMS: 398 (M+H)⁺.

### EXAMPLE 9

### 3-{[(4-Chlorophenyl)methyl]amino}-2-(5-methoxypyridin-3-yl)-3H,4H-pyrido[2,3-d]pyrimidin-4-one

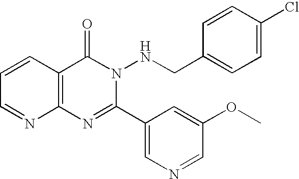

3-{[(4-Chlorophenyl)methyl]amino}-2-(5-methoxypyridin-3-yl)-3H,4H-pyrido[2,3-d]pyrimidin-4-one was synthesized as described in EXAMPLE 1 using 5-methoxynicotinoyl chloride (for synthesis of 5-methoxynicotinoyl chloride, see Khanna, I. K., et. al.; J. Med. Chem.; 43; 16; 2000; 3168-3185) in step 1, and 1-(bromomethyl)-4-chlorobenzene in step 4. ¹H NMR (400 MHz, DMSO-d₆, HCl salt) δ 9.20-9.10 (m, 2H), 8.84 (s, 1H), 8.70 (s, 1H), 8.21 (s, 1H), 7.96 (m, 1H), 7.25 (t, 1H), 7.15 (d, 2H), 6.92 (d, 2H), 4.10 (s, 3H), 4.06 (bs, 2H). LCMS: 394 (M+H)⁺.

### EXAMPLE 10

### 3-{[(4-Chlorophenyl)methyl]amino}-2-(4-fluorophenyl)-3H,4H-pyrido[2,3-d]pyrimidin-4-one

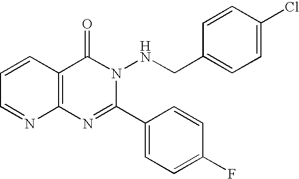

3-{[(4-Chlorophenyl)methyl]amino}-2-(4-fluorophenyl)-3H,4H-pyrido[2,3-d]pyrimidin-4-one was synthesized as described in EXAMPLE 1 using 4-fluorobenzoyl chloride in step 1, and 1-(bromomethyl)-4-chlorobenzene in step 4. ¹H NMR (400 MHz, CDCl₃) δ 9.02 (d, 1H), 8.63 (d, 1H), 7.99 (dd, 2H), 7.49 (dd, 1H), 7.19 (d, 2H), 7.17 (d, 2H), 6.90 (d, 2H), 6.04 (t, 1H), 3.80 (brd, 2H). LCMS: 381 (M+H)⁺.

### EXAMPLE 11

### 2-(4-Fluorophenyl)-3-({[4-(trifluoromethyl)phenyl]methyl}amino)-3H,4H-pyrido[2,3-d]pyrimidin-4-one

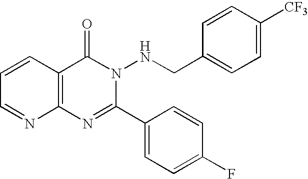

2-(4-Fluorophenyl)-3-({[4-(trifluoromethyl)phenyl]methyl}amino)-3H,4H-pyrido[2,3-d]pyrimidin-4-one was synthesized as described in EXAMPLE 1 using 4-fluorobenzoyl chloride in step 1, and 1-(bromomethyl)-4-(trifluoromethyl)benzene in step 4. ¹H NMR (400 MHz, CDCl₃) δ 9.02 (d, 1H), 8.63 (d, 1H), 7.92 (dd, 2H), 7.46 (dd, 2H), 7.42 (d, 1H), 7.12 (d, 2H), 7.09 (d, 2H), 6.14 (t, 1H), 3.90 (brd, 2H). LCMS: 415 (M+H)⁺.

### EXAMPLE 12

### 3-{[(3,4-Difluorophenyl)methyl]amino}-2-(4-fluorophenyl)-3H,4H-pyrido[2,3-d]pyrimidin-4-one

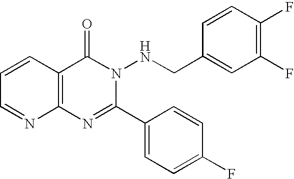

3-{[(3,4-Difluorophenyl)methyl]amino}-2-(4-fluorophenyl)-3H,4H-pyrido[2,3-d]pyrimidin-4-one was synthesized as described in EXAMPLE 1 using 4-fluorobenzoyl chloride in step 1, and 4-(bromomethyl)-1,2-difluorobenzene in step 4. ¹H NMR (400 MHz, CDCl₃) δ 9.02 (d, 1H), 8.63 (d, 1H), 7.99 (dd, 2H), 7.46 (dd, 1H), 7.19 (dd, 2H), 6.99 (dd, 1H), 6.80 (dd, 1H), 6.70 (dd, 1H), 6.03 (t, 1H), 3.80 (brd, 2H). LCMS: 383 (M+H)⁺.

### EXAMPLE 13

### 3-{[(4-Bromophenyl)methyl]amino}-2-(3-methoxy-4-methylphenyl)-3H,4H-pyrido[2,3-d]pyrimidin-4-one

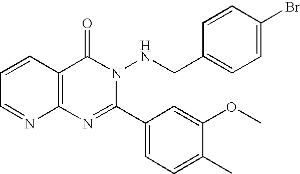

3-{[(4-Bromophenyl)methyl]amino}-2-(3-methoxy-4-methylphenyl)-3H,4H-pyrido[2,3-d]pyrimidin-4-one was synthesized as described in EXAMPLE 1 using 3-methoxy-4-methylbenzoyl chloride in step 1, and 1-bromo-4-(bromomethyl)benzene in step 4. ¹H NMR (400 MHz, CDCl₃) δ 9.08 (d, 1H), 8.00 (d, 1H), 7.52 (dd, 2H), 7.44 (dd, 2H), 7.31 (dd, 2H), 6.84 (d, 2H), 6.20 (t, 1H), 3.84 (s, 3H), 3.80 (brd, 2H), 2.41 (s, 3H). LCMS: 452 (M+H)⁺.

### EXAMPLE 14

### 3-{[(4-Chlorophenyl)methyl]amino}-2-(3-methoxy-4-methylphenyl)-3H,4H-pyrido[2,3-d]pyrimidin-4-one

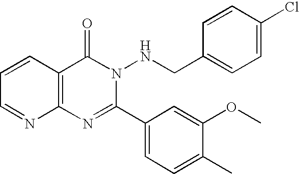

3-{[(4-Chlorophenyl)methyl]amino}-2-(3-methoxy-4-methylphenyl)-3H,4H-pyrido[2,3-d]pyrimidin-4-one was synthesized as described in EXAMPLE 1 using 3-methoxy-4-methylbenzoyl chloride in step 1, and 1-(bromomethyl)-4-chlorobenzene in step 4. ¹H NMR (400 MHz, CDCl₃) δ 9.08 (d, 1H), 8.00 (d, 1H), 7.50 (dd, 2H), 7.40 (dd, 2H), 7.31 (dd, 2H), 6.84 (d, 2H), 6.20 (t, 1H), 3.84 (s, 3H), 3.80 (brd, 2H), 2.39 (s, 3H). LCMS: 407 (M+H)⁺.

### EXAMPLE 15

### 3-{[(4-Chlorophenyl)methyl]amino}-2-(4-hydroxy-3-methoxyphenyl)-3H,4H-pyrido[2,3-d]pyrimidin-4-one

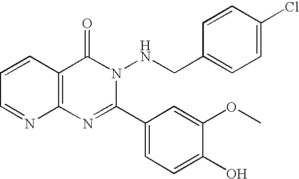

3-{[(4-Chlorophenyl)methyl]amino}-2-(4-hydroxy-3-methoxyphenyl)-3H,4H-pyrido[2,3-d]pyrimidin-4-one was synthesized as described in EXAMPLE 1 using 4-(tert-butyldimethylsilyloxy)-3-methoxybenzoyl chloride (for a procedure to synthesize 4-(tert-butyldimethylsilyloxy)-3-methoxybenzoyl chloride, see Trova, M. P., et. al.; J. Med. Chem. 36; 5; 1993; 580-590) in step 1, and 1-(bromomethyl)-4-chlorobenzene in step 4. The tert-butyldimethylsilyloxy protecting group is cleaved under the conditions in step 3. ¹H NMR (400 MHz, CDCl₃) δ 8.92 (dd, 1H), 8.52 (d, 1H), 7.62 (dd, 1H), 7.34 (dd, 2H), 7.14 (dd, 2H), 6.84 (dd, 2H), 6.82 (d, 1H), 6.04 (t, 1H), 5.12 (s, 1H), 3.82 (s, 3H), 3.70 (brd, 2H). LCMS: 409 (M+H)⁺.

### EXAMPLE 16

### 3-{[(4-Chlorophenyl)methyl]amino}-2-(5-methoxypyridin-3-yl)-3H,4H-pyrido[3,4-d]pyrimidin-4-one

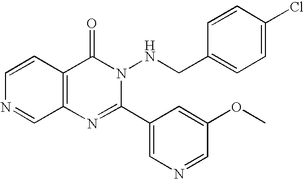

3-{[(4-Chlorophenyl)methyl]amino}-2-(5-methoxypyridin-3-yl)-3H,4H-pyrido[3,4-d]pyrimidin-4-one was synthesized as described in EXAMPLE 1 using 3-aminoisonicotinic acid and 5-methoxynicotinoyl chloride (for synthesis of 5-methoxynicotinoyl chloride, see Khanna, I. K., et. al.; J. Med. Chem.; 43; 16; 2000; 3168-3185) in step 1, and 1-(bromomethyl)-4-chlorobenzene in step 4. ¹H NMR (400 MHz, DMSO-d₆, HCl salt) δ 9.12 (s, 1H), 8.75 (d, 1H), 8.44 (d, 1H), 8.42 (d, 1H), 8.08 (d, 1H), 7.54 (m, 1H), 7.15-7.12 (m, 2H), 6.99 (bs, 1H), 6.83-6.80 (m, 2H), 3.95 (bs, 2H), 3.85 (s, 3H). LCMS: 394 (M+H)⁺.

### EXAMPLE 17

### 3-{[(4-Bromophenyl)methyl]amino}-2-(4-fluorophenyl)-3H,4H-pyrido[3,4-d]pyrimidin-4-one

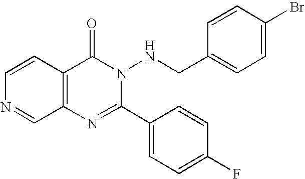

3-{[(4-Bromophenyl)methyl]amino}-2-(4-fluorophenyl)-3H,4H-pyrido[3,4-d]pyrimidin-4-one was synthesized as described in EXAMPLE 1 using 3-aminoisonicotinic acid and 4-fluorobenzoyl chloride in step 1, and 1-bromo-4-(bromomethyl)benzene in step 4. ¹H NMR (400 MHz, CDCl₃) δ 9.21 (s, 1H), 8.74 (d, 1H), 8.09-8.07 (m, 1H), 7.87-7.82 (m, 2H), 7.32 (d, 2H), 7.19-7.15 (m, 2H), 6.82 (d, 2H), 6.05 (t, 1H), 3.77 (m, 2H). LCMS: 428 (M+H)⁺.

### EXAMPLE 18

### 3-{[(4-Chlorophenyl)methyl]amino}-2-(5-chloropyridin-3-yl)-3H,4H-pyrido[3,4-d]pyrimidin-4-one

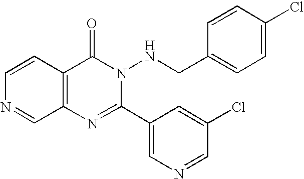

3-{[(4-Chlorophenyl)methyl]amino}-2-(5-chloropyridin-3-yl)-3H,4H-pyrido[3,4-d]pyrimidin-4-one was synthesized as described in EXAMPLE 1 using 3-aminoisonicotinic acid in step 1, and 1-(bromomethyl)-4-chlorobenzene in step 4. ¹H NMR (400 MHz, CDCl₃) δ 9.22 (s, 1H), 8.98 (s, 1H), 8.80 (d, 1H), 8.64 (s, 1H), 8.22 (d, 1H), 7.99 (s, 1H), 7.18 (d, 2H), 6.81 (d, 2H), 6.12 (t, 1H), 3.88 (brd, 2H). LCMS: 399 (M+H)⁺.

### EXAMPLE 19

### 3-{[(4-Chlorophenyl)methyl]amino}-2-(3-methyl-4-nitrophenyl)-3H,4H-pyrido[3,4-d]pyrimidin-4-one

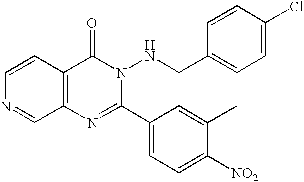

3-{[(4-Chlorophenyl)methyl]amino}-2-(3-methyl-4-nitrophenyl)-3H,4H-pyrido[3,4-d]pyrimidin-4-one was synthesized as described in EXAMPLE 1 using 3-aminoisonicotinic acid and 3-methyl-4-nitrobenzoyl chloride in step 1, and 1-(bromomethyl)-4-chlorobenzene in step 4. ¹H NMR (400 MHz, CDCl₃) δ 9.20 (d, 1H), 8.63 (dd, 1H), 8.02 (dd, 1H), 7.63 (s, 1H), 7.50 (s, 1H), 7.20 (dd, 2H), 7.00 (dd, 2H), 6.71 (d, 1H), 6.16 (t, 1H), 3.80 (brd, 2H), 2.24 (s, 3H). LCMS: 422 (M+H)⁺.

### EXAMPLE 20

### 2-(4-Amino-3-methylphenyl)-3-{[(4-chlorophenyl)methyl]amino}-3H,4H-pyrido[3,4-d]pyrimidin-4-one

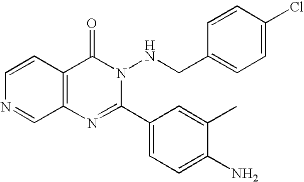

To 3-{[(4-chlorophenyl)methyl]amino}-2-(3-methyl-4-nitrophenyl)-3H,4H-pyrido[3,4-d]pyrimidin-4-one (30 mg, for synthesis see EXAMPLE 19) in ethanol (4 mL) was added 10 mg of palladium (10% on carbon). The reaction was under a hydrogen atmosphere for 4 h. The reaction mixture was diluted with ethyl acetate (10 mL) then filtered through a plug of Celite. The organics were concentrated to dryness then recrystallized using cold hexane to afford 2-(4-amino-3-methylphenyl)-3-{[(4-chlorophenyl)methyl]amino}-3H,4H-pyrido[3,4-d]pyrimidin-4-one (24 mg, 87% yield). ¹H NMR (400 MHz, CDCl₃) δ 9.20 (d, 1H), 8.63 (dd, 1H), 8.02 (dd, 1H), 7.63 (s, 1H), 7.50 (s, 1H), 7.20 (dd, 2H), 7.00 (dd, 2H), 6.71 (d, 1H), 6.16 (t, 1H), 3.88 (brs, 2H), 3.80 (brd, 2H), 2.24 (s, 3H). LCMS: 392 (M+H)⁺.

### EXAMPLE 21

### 3-{[(4-Chlorophenyl)methyl]amino}-2-(4-hydroxy-3-methylphenyl)-3H,4H-pyrido[3,4-d]pyrimidin-4-one

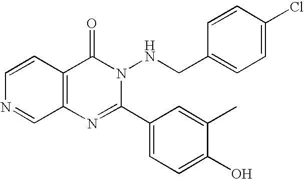

3-{[(4-Chlorophenyl)methyl]amino}-2-(4-hydroxy-3-methylphenyl)-3H,4H-pyrido[3,4-d]pyrimidin-4-one was synthesized as described in EXAMPLE 1 using 3-aminoisonicotinic acid and 4-(tert-butyldimethylsilyloxy)-3-methylbenzoyl chloride (for a procedure to synthesize 4-(tert-butyldimethylsilyloxy)-3-methylbenzoyl chloride, see Trova, M. P., et. al.; J. Med. Chem. 36; 5; 1993; 580-590) in step 1, and 1-(bromomethyl)-4-chlorobenzene in step 4. The tert-butyldimethylsilyloxy protecting group is cleaved under the conditions in step 3. ¹H NMR (400 MHz, CDCl₃) δ 9.10 (d, 1H), 8.60 (d, 1H), 7.98 (d, 1H), 7.60 (d, 1H), 7.56 (s, 1H), 7.30 (s, 1H), 7.18 (dd, 2H), 6.81 (dd, 2H), 6.00 (t, 1H), 5.02 (brs, 1H), 3.68 (brd, 2H), 2.22 (s, 3H). LCMS: 393 (M+H)⁺.

### EXAMPLE 22

### 3-{[(4-Chlorophenyl)methyl]amino}-2-(4-hydroxy-3-methoxyphenyl)-3H,4H-pyrido[3,4-d]pyrimidin-4-one

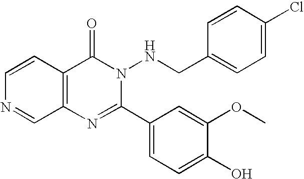

3-{[(4-Chlorophenyl)methyl]amino}-2-(4-hydroxy-3-methoxyphenyl)-3H,4H-pyrido[3,4-d]pyrimidin-4-one was synthesized as described in EXAMPLE 1 using 3-aminoisonicotinic acid and 4-(tert-butyldimethylsilyloxy)-3-methoxybenzoyl chloride (for a procedure to synthesize 4-(tert-butyldimethylsilyloxy)-3-methoxybenzoyl chloride, see Trova, M. P., et. al.; J. Med. Chem. 36; 5; 1993; 580-590) in step 1, and 1-(bromomethyl)-4-chlorobenzene in step 4. The tert-butyldimethylsilyloxy protecting group is cleaved under the conditions in step 3. ¹H NMR (400 MHz, CDCl₃) δ 9.10 (d, 1H), 8.60 (d, 1H), 7.98 (d, 1H), 7.44 (d, 1H), 7.40 (s, 1H), 7.28 (s, 1H), 7.08 (dd, 2H), 6.84 (dd, 2H), 6.03 (t, 1H), 5.82 (brs, 1H), 3.82 (s, 3H), 3.68 (brd, 2H). LCMS: 409 (M+H)⁺.

### EXAMPLE 23

### 3-{[(4-Chlorophenyl)methyl]amino}-2-(4-fluorophenyl)-3H,4H-pyrido[4,3-d]pyrimidin-4-one

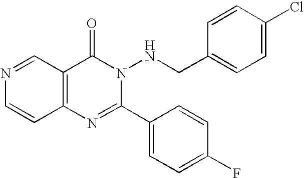

3-{[(4-Chlorophenyl)methyl]amino}-2-(4-fluorophenyl)-3H,4H-pyrido[4,3-d]pyrimidin-4-one was synthesized as described in EXAMPLE 1 using 4-aminonicotinic acid and 4-fluorobenzoyl chloride in step 1, and 1-(bromomethyl)-4-chlorobenzene in step 4. ¹H NMR (400 MHz, CDCl₃) δ 9.49 (s, 1H), 8.80 (d, 1H), 7.79-7.75 (m, 2H), 7.50 (d, 1H), 7.11-7.09 (m, 4H), 6.83-6.80 (m, 2H), 5.99 (t, 1H), 3.73 (bs, 2H). LCMS: 381 (M+H)⁺.

### EXAMPLE 24

### 3-{[(4-Chlorophenyl)methyl]amino}-2-(5-methoxypyridin-3-yl)-3H,4H-pyrido[3,2-d]pyrimidin-4-one

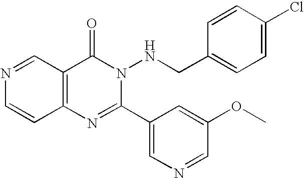

3-{[(4-Chlorophenyl)methyl]amino}-2-(5-methoxypyridin-3-yl)-3H,4H-pyrido[3,2-d]pyrimidin-4-one was synthesized as described in EXAMPLE 1 using 3-aminopicolinic acid and 5-methoxynicotinoyl chloride (for synthesis of 5-methoxynicotinoyl chloride, see Khanna, I. K., et. al.; J. Med. Chem.; 43; 16; 2000; 3168-3185) in step 1, and 1-(bromomethyl)-4-chlorobenzene in step 4. ¹H NMR (400 MHz, DMSO-d₆, HCl salt) δ 9.11 (dd, 1H), 8.74 (dd, 1H), 8.58 (m, 2H), 7.76-7.70 (m, 2H), 7.22 (d, 2H), 7.06 (bs, 1H), 6.90 (d, 2H), 4.03 (bs, 2H), 3.96 (s, 3H). LCMS: 394 (M+H)⁺.

### EXAMPLE 25

### 3-{[(4-Bromophenyl)methyl]amino}-2-(4-fluorophenyl)-3H,4H-pyrido[3,2-d]pyrimidin-4-one

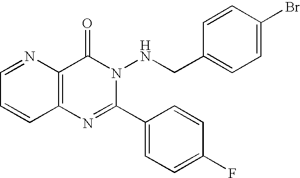

3-{[(4-Bromophenyl)methyl]amino}-2-(4-fluorophenyl)-3H,4H-pyrido[3,2-d]pyrimidin-4-one was synthesized as described in EXAMPLE 1 using 3-aminopicolinic acid and 4-fluorobenzoyl chloride in step 1, and 1-bromo-4-(bromomethyl)benzene in step 4. ¹H NMR (400 MHz, CDCl₃) δ 9.07-9.06 (m, 1H), 8.70 (dd, 1H), 7.99-7.55 (m, 2H), 7.54 (dd, 1H), 7.34-7.31 (m, 2H), 7.17-7.13 (m, 2H), 6.84 (d, 2H), 3.78 (m, 2H). LCMS: 425 (M+H)⁺.

### EXAMPLE 26

### 3-{[(4-Bromophenyl)methyl]amino}-2-(4-hydroxyphenyl)-3H,4H-thieno[2,3-d]pyrimidin-4-one

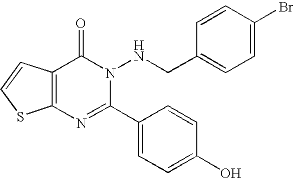

3-{[(4-Bromophenyl)methyl]amino}-2-(4-hydroxyphenyl)-3H,4H-thieno[2,3-d]pyrimidin-4-one was synthesized as described in EXAMPLE 6 using 2-aminothiophene-3-carboxylic acid, 4-methoxybenzoyl chloride, and 1-bromo-4-(bromomethyl)benzene as starting materials. ¹H NMR (400 MHz, CDCl₃) δ 7.80 (d, 1H), 7.50 (dd, 2H), 7.44 (dd, 2H), 7.31 (d, 1H), 7.20 (d, 2H), 6.90 (d, 2H), 6.17 (t, 1H), 5.22 (s, 1H), 3.78 (brd, 2H). LCMS: 429 (M+H)⁺.

### EXAMPLE 27

### 3-{[(4-Chlorophenyl)methyl]amino}-2-(5-methoxypyridin-3-yl)-3H,4H-thieno[3,2-d]pyrimidin-4-one

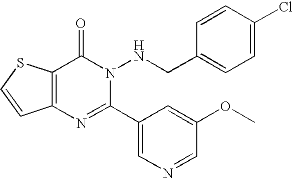

3-{[(4-Chlorophenyl)methyl]amino}-2-(5-methoxypyridin-3-yl)-3H,4H-thieno[3,2-d]pyrimidin-4-one was synthesized as described in EXAMPLE 1 using 3-aminothiophene-2-carboxylic acid and 5-methoxynicotinoyl chloride (for synthesis of 5-methoxynicotinoyl chloride, see Khanna, I. K., et. al.; J. Med. Chem.; 43; 16; 2000; 3168-3185) in step 1, and 1-(bromomethyl)-4-chlorobenzene in step 4. ¹H NMR (400 MHz, DMSO-d₆, HCl salt) δ 8.43 (m, 2H), 8.28 (m, 1H), 7.55 (s, 1H), 7.44 (m, 1H), 7.15 (d, 2H), 6.93 (bs, 1H), 6.79 (d, 2H), 3.95 (bs, 2H), 3.85 (s, 3H). LCMS: 399 (M+H)⁺.

### EXAMPLE 28

### 3-[(Cyclobutylmethyl)amino]-2-(5-methoxypyridin-3-yl)-3H,4H-thieno[3,2-d]pyrimidin-4-one

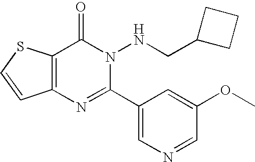

3-[(Cyclobutylmethyl)amino]-2-(5-methoxypyridin-3-yl)-3H,4H-thieno[3,2-d]pyrimidin-4-one was synthesized as described in EXAMPLE 1 using 3-aminothiophene-2-carboxylic acid and 5-methoxynicotinoyl chloride (for synthesis of 5-methoxynicotinoyl chloride, see Khanna, I. K., et. al.; J. Med. Chem.; 43; 16; 2000; 3168-3185) in step 1, and (bromomethyl)cyclobutane in DMF at 90° C. for 1 h in step 4. ¹H NMR (400 MHz, CD₃OD, TFA salt) δ 8.82 (s, 1H), 8.54 (s, 1H), 8.33 (s, 1H), 8.13 (d, 1H), 7.41 (d, 1H), 4.04 (s, 3H), 2.84 (bs, 2H), 2.26 (m, 1H), 1.90-1.70 (m, 4H), 1.43 (bs, 2H). LCMS: 343 (M+H)⁺.

### EXAMPLE 29

### 3-{[(4-Chlorophenyl)methyl]amino}-2-(5-methoxypyridin-3-yl)-3,4-dihydropteridin-4-one

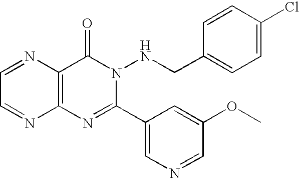

3-{[(4-Chlorophenyl)methyl]amino}-2-(5-methoxypyridin-3-yl)-3,4-dihydropteridin-4-one was synthesized as described in EXAMPLE 1 using 3-aminopyrazine-2-carboxylic acid and 5-methoxynicotinoyl chloride (for synthesis of 5-methoxynicotinoyl chloride, see Khanna, I. K., et. al.; J. Med. Chem.; 43; 16; 2000; 3168-3185) in step 1, and 1-(bromomethyl)-4-chlorobenzene with DMF as the solvent in step 4. ¹H NMR (400 MHz, CD₃OD, HCl salt) δ 9.09 (s, 1H), 8.97 (s, 1H), 8.75 (s, 1H), 8.63 (s, 1H), 8.14 (s, 1H), 7.13 (d, 2H), 6.89 (d, 2H), 4.07 (s, 3H), 4.03 (bs, 2H). LCMS: 395 (M+H)⁺.

### EXAMPLE 30

### 3-{[(4-Chlorophenyl)methyl]amino}-2-(4-fluorophenyl)-3H,4H-pyrimido[4,5-d][1,3]diazin-4-one

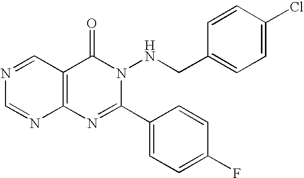

3-{[(4-Chlorophenyl)methyl]amino}-2-(4-fluorophenyl)-3H,4H-pyrimido[4,5-d][1,3]diazin-4-one was synthesized as described in EXAMPLE 42 using 4-aminopyrimidine-5-carboxylic acid and 4-fluorobenzoyl chloride in step 1, and 1-(bromomethyl)-4-chlorobenzene with DMF as the solvent in step 4. ¹H NMR (400 MHz, CD₃OD, TFA salt) δ 9.52 (s, 1H), 9.29 (s, 1H), 7.62 (dd, 2H), 7.09 (t, 2H), 7.03 (d, 2H), 6.79 (d, 2H), 3.78 (bs, 2H). LCMS: 382 (M+H)⁺.

### EXAMPLE 31

### 3-{[(4-Bromophenyl)methyl]amino}-2-(4-fluorophenyl)-3H,4H-thieno[2,3-d]pyrimidin-4-one

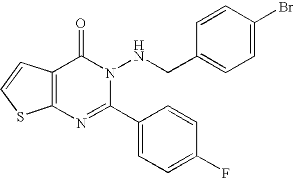

### Step 1: Ethyl 2-(4-fluorobenzamido)thiophene-3-carboxylate

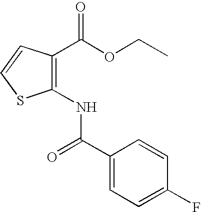

To a stirred solution of ethyl 2-aminothiophene-3-carboxylate (10 g) in THF (150 mL), was added 4-fluorobenzoyl chloride (10.2 g). Then TEA (12 mL) was added dropwise. After 3 h at RT, the mixture was filtered and the filtrate was concentrated to give ethyl 2-(4-fluorobenzamido)thiophene-3-carboxylate (13.5 g, 74%) as a yellow solid. LCMS: 294 (M+H)⁺.

### Step 2: 2-(4-Fluorobenzamido)thiophene-3-carboxylic Acid

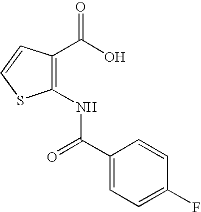

To a stirred solution of ethyl 2-aminothiophene-3-carboxylate (5 g) in THF (20 mL), was added solution of a lithium hydroxide (1.1 g) in water (20 mL). A 5 h at 75° C., water (100 mL) is added, and the pH adjusted to 6 with 2N HCl. The precipitate is filtered and washed with water (3×10 mL) to give 2-(4-fluorobenzamido)thiophene-3-carboxylic acid (3.2 g, 65%) as a gray solid. LCMS: 266 (M+H)⁺.

### Step 3: 2-(4-Fluorophenyl)-4H-thieno[2,3-d][1,3]oxazin-4-one

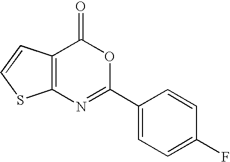

A solution of 2-(4-fluorobenzamido)thiophene-3-carboxylic acid (2 g) in acetic anhydride (10 mL) was stirred for 3 h at 140° C. The mixture is concentrated to give 2-(4-fluorophenyl)-4H-thieno[2,3-d][1,3]oxazin-4-one (1.5 g, 64%) as a gray solid. LCMS: 248 (M+H)⁺.

### Step 4: 3-Amino-2-(4-fluorophenyl)thieno[2,3-d]pyrimidin-4(3H)-one

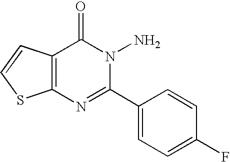

To a stirred solution of 2-(4-fluorophenyl)-4H-thieno[2,3-d][1,3]oxazin-4-one (900 mg) in acetic acid (10 mL), was added hydrazine monohydrate (1 mL). After heating at reflux for 16 h, the precipitate is filtered, dissolved in polyphosphoric acid (5 g), and heated for 2 h at 140° C. Ice water (10 mL) is added. The precipitate is filtered and washed with water (3×10 mL). Purification by silica gel chromatography gave 3-amino-2-(4-fluorophenyl)thieno[2,3-d]pyrimidin-4(3H)-one (389 mg, 41%) as a brown solid. NMR (300 MHz, DMSO-d₆) δ 7.88 (m, 2H), 7.63 (d, 1H), 7.45 (d, 1H), 7.32 (t, 2H), 5.69 (s, 2H). LCMS: 262 (M+H)⁺.

### Step 5: 3-{[(4-Bromophenyl)methyl]amino}-2-(4-fluorophenyl)-3H,4H-thieno[2,3-d]pyrimidin-4-one

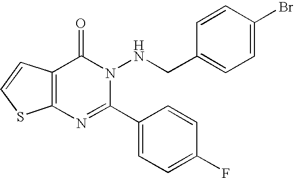

To a solution of 3-amino-2-(4-fluorophenyl)thieno[2,3-d]pyrimidin-4(3H)-one (24 mg) in THF (0.5 mL), was added sodium hydride (7 mg, 60%) and 1-bromo-4-(bromomethyl)benzene (25 mg). After 3 h at RT, the reaction mixture is concentrated to residue. Purification by silica gel chromatography (100% DCM as the eluting solvent) gave 3-{[(4-bromophenyl)methyl]amino}-2-(4-fluorophenyl)-3H,4H-thieno[2,3-d]pyrimidin-4-one. ¹H NMR (400 MHz, CDCl₃) δ 7.83-7.80 (m, 2H), 7.51 (d, 1H), 7.31-7.29 (m, 3H), 7.16-7.11 (m, 2H), 6.84 (d, 2H), 6.12 (t, 1H), 3.77 (m, 2H). LCMS: 427 (M+H)⁺.

### EXAMPLE 32

### 3-{[(4-Chlorophenyl)methyl]amino}-2-(4-fluorophenyl)-3H,4H-thieno[2,3-d]pyrimidin-4-one

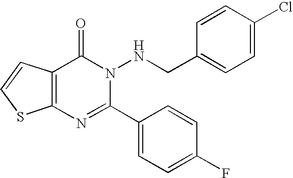

3-{[(4-Chlorophenyl)methyl]amino}-2-(4-fluorophenyl)-3H,4H-thieno[2,3-d]pyrimidin-4-one was synthesized as described in EXAMPLE 31 using 1-(bromomethyl)-4-chlorobenzene in step 5. ¹H NMR (400 MHz, CDCl₃) δ 7.84-7.81 (m, 2H), 7.51 (d, 1H), 7.30 (d, 1H), 7.18-7.11 (m, 4H), 6.90 (d, 2H), 6.12 (t, 1H), 3.77 (m, 2H). LCMS: 386 (M+H)⁺.

### EXAMPLE 33

### 3-{[(3,4-Dichlorophenyl)methyl]amino}-2-(4-fluorophenyl)-3H,4H-thieno[2,3-d]pyrimidin-4-one

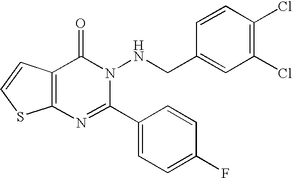

3-{[(3,4-Dichlorophenyl)methyl]amino}-2-(4-fluorophenyl)-3H,4H-thieno[2,3-d]pyrimidin-4-one was synthesized as described in EXAMPLE 31 using 4-(bromomethyl)-1,2-dichlorobenzene in step 5. ¹H NMR (400 MHz, CDCl₃) δ 7.80-7.60 (m, 2H), 7.51 (d, 1H), 7.30 (d, 1H), 7.26 (d, 1H), 7.16-7.11 (m, 2H), 7.00 (d, 1H), 6.80-6.77 (m, 1H), 6.12 (t, 1H), 3.78 (m, 2H). LCMS: 424 (M+H)⁺.

### EXAMPLE 34

### 3-{[(4-Chlorophenyl)methyl]amino}-2-(5-methoxypyridin-3-yl)-3H,4H-thieno[2,3-d]pyrimidin-4-one

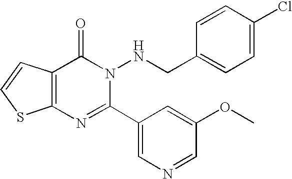

3-{[(4-Chlorophenyl)methyl]amino}-2-(5-methoxypyridin-3-yl)-3H,4H-thieno[2,3-d]pyrimidin-4-one was synthesized as described in EXAMPLE 31 using 5-methoxynicotinoyl chloride (for synthesis of 5-methoxynicotinoyl chloride, see Khanna, I. K., et. al.; J. Med. Chem.; 43; 16; 2000; 3168-3185) in step 1, and 1-(bromomethyl)-4-chlorobenzene in step 5. LCMS: 399 (M+H)⁺.

### EXAMPLE 35

### 3-{[(4-Bromophenyl)methyl]amino}-2-(4-fluorophenyl)-3H,4H-thieno[3,2-d]pyrimidin-4-one

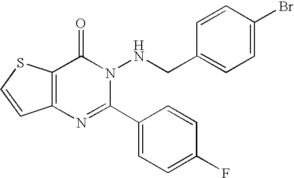

3-{[(4-Bromophenyl)methyl]amino}-2-(4-fluorophenyl)-3H,4H-thieno[3,2-d]pyrimidin-4-one was synthesized as described in EXAMPLE 31 using methyl 3-aminothiophene-2-carboxylate and 4-fluorobenzoyl chloride in step 1, and 1-bromo-4-(bromomethyl)benzene in step 5. ¹H NMR (400 MHz, CDCl₃) δ 7.81 (d, 1H), 7.79 (dd, 2H), 7.39 (d, 1H), 7.37 (d, 2H), 7.17 (dd, 2H), 6.81 (d, 2H), 6.03 (t, 1H), 3.79 (brd, 2H). LCMS: 431 (M+H)⁺.

### EXAMPLE 36

### 3-{[(3,4-Dichlorophenyl)methyl]amino}-2-(4-fluorophenyl)-3H,4H-thieno[3,2-d]pyrimidin-4-one

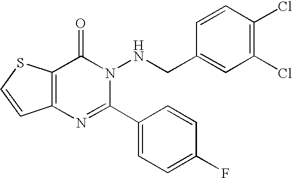

3-{[(3,4-Dichlorophenyl)methyl]amino}-2-(4-fluorophenyl)-3H,4H-thieno[3,2-d]pyrimidin-4-one was synthesized as described in EXAMPLE 31 using methyl 3-aminothiophene-2-carboxylate and 4-fluorobenzoyl chloride in step 1, and 4-(bromomethyl)-1,2-dichlorobenzene in step 5. ¹H NMR (400 MHz, CDCl₃) δ 7.82 (d, 1H), 7.77 (dd, 2H), 7.39 (d, 1H), 7.25 (dd, 1H), 7.18 (dd, 2H), 6.99 (s, 1H), 6.79 (dd, 1H), 6.08 (t, 1H), 3.81 (brd, 2H). LCMS: 421 (M+H)⁺.

### EXAMPLE 37

### 3-{[(4-Chlorophenyl)methyl]amino}-2-(4-fluorophenyl)-3H,4H-thieno[3,2-d]pyrimidin-4-one

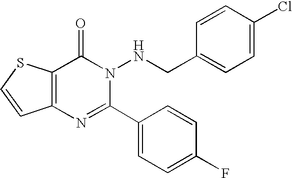

3-{[(4-Chlorophenyl)methyl]amino}-2-(4-fluorophenyl)-3H,4H-thieno[3,2-d]pyrimidin-4-one was synthesized as described in EXAMPLE 31 using methyl 3-aminothiophene-2-carboxylate and 4-fluorobenzoyl chloride in step 1, and 1-(bromomethyl)-4-chlorobenzene in step 5. ¹H NMR (400 MHz, CDCl₃) δ 7.82 (d, 1H), 7.79 (dd, 2H), 7.38 (d, 1H), 7.18 (d, 2H), 7.16 (dd, 2H), 6.89 (d, 2H), 6.07 (t, 1H), 3.80 (brd, 2H). LCMS: 486 (M+H)⁺.

### EXAMPLE 38

### 1-{[(4-Bromophenyl)methyl]amino}-2-(4-fluorophenyl)-7-methyl-6,7-dihydro-1H-purin-6-one

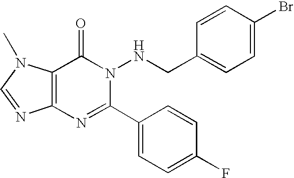

### Step 1: 5-(4-Fluorophenyl)-1-methylimidazo[4,5-d][1,3]oxazin-7(1H)-one

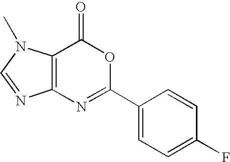

To a solution of ethyl 4-amino-1-methyl-1H-imidazole-5-carboxylate (2 g, for a synthesis of ethyl 4-amino-1-methyl-1H-imidazole-5-carboxylate, see Mann, P.; J. Chem. Soc. 1945; 751-757) in acetonitrile (80 mL), was added triphenylphosphine (4.2 g), hexachloroethane (4 g) and TEA (5 mL). After 90 minutes at reflux, 4-fluorobenzoyl chloride (4.3 g) was added followed by pyridine (40 mL). After an additional 1 h at reflux, the mixture was concentrated. Purification by silica gel chromatography (20% to 50% ethyl acetate/petroleum ether) gave 5-(4-fluorophenyl)-1-methylimidazo[4,5-d][1,3]oxazin-7(1H)-one (750 mg, 26%) as a light yellow solid. LCMS: 246 (M+H)⁺.

### Step 2: 4-Fluoro-N-(5-(hydrazinecarbonyl)-1-methyl-1H-imidazol-4-yl)benzamide

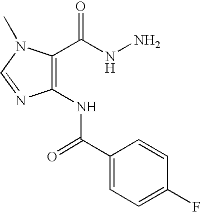

To a solution of 5-(4-fluorophenyl)-1-methylimidazo[4,5-d][1,3]oxazin-7(1H)-one (700 mg) in THF (30 mL), was added hydrazine (340 mg) dropwise with stirring, while at a temperature of 0° C. After 1 h at 0° C., the mixture was concentrated. Purification by recrystallization from 33% ethyl acetate/petroleum ether gave 4-fluoro-N-(5-(hydrazinecarbonyl)-1-methyl-1H-imidazol-4-yl)benzamide (650 mg, 82%) as a light yellow solid. LCMS: 278 (M+H)⁺.

### Step 3: 1-Amino-2-(4-fluorophenyl)-7-methyl-1H-purin-6(7H)-one

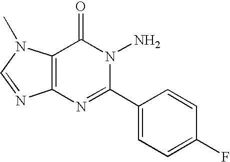

To a solution of 4-fluoro-N-(5-(hydrazinecarbonyl)-1-methyl-1H-imidazol-4-yl)benzamide (600 mg) in toluene (150 mL), was added 4-methylbenzenesulfonic acid (2 g). After 5 h at reflux, the mixture was concentrated. Purification by silica gel chromatography (1% to 3% methanol/DCM), followed by a recrystallization from 33% ethyl acetate/hexane gave 1-amino-2-(4-fluorophenyl)-7-methyl-1H-purin-6(7H)-one (102 mg, 16%) as an off-white solid. ¹H NMR (300 MHz, DMSO-d₆) δ 8.22 (s, 1H), 7.80 (m, 2H), 7.29 (m, 2H), 5.60 (s, 2H), 4.01 (s, 3H). LCMS: 260 (M+H)⁺.

### Step 4: 1-{[(4-Bromophenyl)methyl]amino}-2-(4-fluorophenyl)-7-methyl-6,7-dihydro-1H-purin-6-one

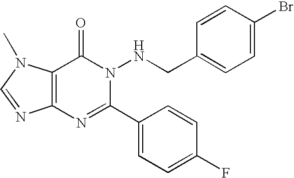

1-{[(4-Bromophenyl)methyl]amino}-2-(4-fluorophenyl)-7-methyl-6,7-dihydro-1H-purin-6-one was synthesized as described in EXAMPLE 1, step 5, using 1-amino-2-(4-fluorophenyl)-7-methyl-1H-purin-6(7H)-one and 1-bromo-4-(bromomethyl)benzene as starting materials. ¹H NMR (400 MHz, CDCl₃) δ 7.84 (s, 1H), 7.81 (dd, 2H), 7.34 (dd, 2H), 7.10 (dd, 2H), 6.80 (dd, 2H), 6.01 (t, 1H), 4.11 (s, 3H), 3.78 (brd, 2H). LCMS: 429 (M+H)⁺.

### EXAMPLE 39

### 1-{[(4-Chlorophenyl)methyl]amino}-2-(4-fluorophenyl)-7-methyl-6,7-dihydro-1H-purin-6-one

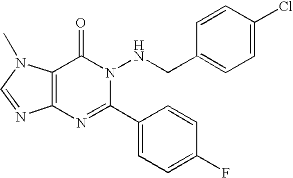

1-{[(4-Chlorophenyl)methyl]amino}-2-(4-fluorophenyl)-7-methyl-6,7-dihydro-1H-purin-6-one was synthesized as described in EXAMPLE 38 using 1-(bromomethyl)-4-chlorobenzene in the final step. ¹H NMR (400 MHz, CDCl₃) δ 7.84 (s, 1H), 7.81 (dd, 2H), 7.18 (dd, 2H), 7.12 (dd, 2H), 6.82 (dd, 2H), 6.01 (t, 1H), 4.13 (s, 3H), 3.78 (brd, 2H). LCMS: 384 (M+H)⁺.

### EXAMPLE 40

### 6-{[(4-Bromophenyl)methyl]amino}-5-(4-fluorophenyl)-1-methyl-1H,6H,7H-pyrazolo[4,3-d]pyrimidin-7-one

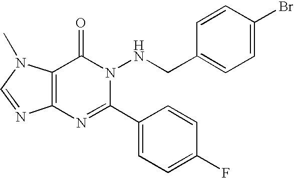

### Step 1: Methyl 1-methyl-4-nitro-1H-pyrazole-5-carboxylate

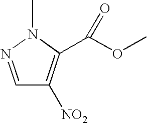

To a solution of 1-methyl-4-nitro-1H-pyrazole-5-carboxylic acid (2 g, for the synthesis of 1-methyl-4-nitro-1H-pyrazole-5-carboxylic acid, see Perevalov, V. P., et. al.; Chem. Het. Compounds 19; 12; 1983; 1326-1330) in methanol (20 mL), was added HCl (g). The resulting solution was allowed to react, with stirring, for 16 h at reflux. The mixture was concentrated to give methyl 1-methyl-4-nitro-1H-pyrazole-5-carboxylate as a yellow oil (1.8 g, 83%). LCMS: 185 (M+H)⁺.

### Step 2: Methyl 4-amino-1-methyl-1H-pyrazole-5-carboxylate

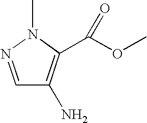

To a solution of methyl 1-methyl-4-nitro-1H-pyrazole-5-carboxylate (1.5 g) in THF (20 mL) was added Pd/C (500 mg, 10%). The solution was stirred under a hydrogen atmosphere for 6 h at RT. The reaction mixture was filtered, dried over sodium sulfate, filtered, and concentrated to give 1.1 g of crude methyl 4-amino-1-methyl-1H-pyrazole-5-carboxylate. LCMS: 156 (M+H)⁺.

### Step 3: Methyl 4-(4-fluorobenzamido)-1-methyl-1H-pyrazole-5-carboxylate

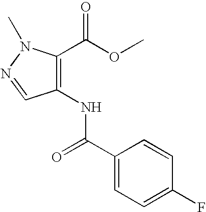

To a solution of methyl 4-amino-1-methyl-1H-pyrazole-5-carboxylate (1 g) in THF (20 mL) was added TEA (1.2 mL) and 4-fluorobenzoyl chloride (1.3 g). After 4 h at RT, the mixture was concentrated to residue, taken up in ethyl acetate (50 mL) washed with sodium bicarbonate solution (50 mL), and washed with NaCl solution (50 mL). The organic layer was dried over sodium sulfate, filtered, and concentrated. Purification by silica gel chromatography (20% ethyl acetate/petroleum ether) gave methyl 4-(4-fluorobenzamido)-1-methyl-1H-pyrazole-5-carboxylate (0.8 g, 45%). LCMS: 278 (M+H)⁺.

### Step 4: 4-Fluoro-N-(5-(hydrazinecarbonyl)-1-methyl-1H-pyrazol-4-yl)benzamide

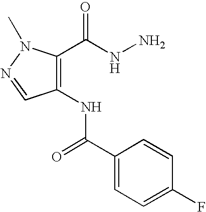

To a solution of methyl 4-(4-fluorobenzamido)-1-methyl-1H-pyrazole-5-carboxylate (800 mg) in methanol (20 mL) was added hydrazine monohydrate (2 g, 80%). After 6 h at RT, the mixture was concentrated. The residue was taken up in THF (50 mL), washed with brine (3×50 mL), dried over sodium sulfate, and concentrated to give 4-fluoro-N-(5-(hydrazinecarbonyl)-1-methyl-1H-pyrazol-4-yl)benzamide (0.6 g, 74%) as a brown solid. LCMS: 278 (M+H)⁺.

### Step 5: 6-Amino-5-(4-fluorophenyl)-1-methyl-1H-pyrazolo[4,3-d]pyrimidin-7(6H)-one

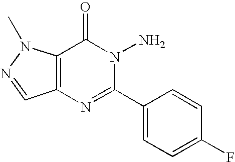

A mixture of 4-fluoro-N-(5-(hydrazinecarbonyl)-1-methyl-1H-pyrazol-4-yl)benzamide (500 mg) and polyphosphoric acid (15 g, 80%) was heated at 110° C. for 1 h. The reaction mixture was then quenched with ice (15 g) and filtered. The filter cake was washed with cold water (3×10 mL). Purification by silica gel chromatography (20% ethyl acetate/petroleum ether) gave 6-amino-5-(4-fluorophenyl)-1-methyl-1H-pyrazolo[4,3-d]pyrimidin-7(6H)-one (105 mg, 22%) as a white solid. ¹H NMR (400 MHz, CDCl₃) δ 7.94 (s, 1H), 7.77 (m, 2H), 7.18 (m, 2H), 4.92 (s, 2H), 4.36 (s, 3H). LCMS: 260 (M+H)⁺.

### Step 6: 6-{[(4-Bromophenyl)methyl]amino}-5-(4-fluorophenyl)-1-methyl-1H,6H,7H-pyrazolo[4,3-d]pyrimidin-7-one

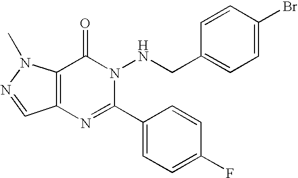

6-{[(4-Bromophenyl)methyl]amino}-5-(4-fluorophenyl)-1-methyl-1H,6H,7H-pyrazolo[4,3-d]pyrimidin-7-one was synthesized as described in EXAMPLE 1, step 4 using 1-bromo-4-(bromomethyl)benzene. ¹H NMR (400 MHz, CDCl₃) δ 7.91 (s, 1H), 7.71 (dd, 2H), 7.34 (d, 2H), 7.12 (dd, 2H), 6.81 (d, 2H), 5.92 (t, 1H), 4.33 (s, 3H), 3.72 (brd, 2H). LCMS: 429 (M+H)⁺.

### EXAMPLE 41

### 6-{[(4-Chlorophenyl)methyl]amino}-5-(4-fluorophenyl)-1-methyl-1H,6H,7H-pyrazolo[4,3-d]pyrimidin-7-one

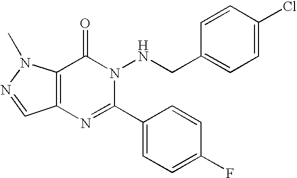

6-{[(4-Chlorophenyl)methyl]amino}-5-(4-fluorophenyl)-1-methyl-1H,6H,7H-pyrazolo[4,3-d]pyrimidin-7-one was synthesized as described in EXAMPLE 40, using 1-(bromomethyl)-4-chlorobenzene as a starting material in the final step. ¹H NMR (400 MHz, CDCl₃) δ 7.91 (s, 1H), 7.72 (dd, 2H), 7.18 (d, 2H), 7.14 (d, 2H), 6.88 (d, 2H), 5.92 (t, 1H), 4.36 (s, 3H), 3.76 (brd, 2H). LCMS: 384 (M+H)⁺.

### EXAMPLE 42

### 6-{[(3,4-Difluorophenyl)methyl]amino}-5-(4-fluorophenyl)-1-methyl-1H,6H,7H-pyrazolo[4,3-d]pyrimidin-7-one

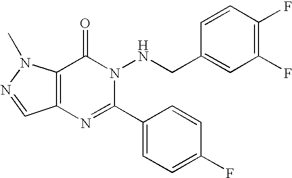

6-{[(3,4-Difluorophenyl)methyl]amino}-5-(4-fluorophenyl)-1-methyl-1H,6H,7H-pyrazolo[4,3-d]pyrimidin-7-one was synthesized as described in EXAMPLE 40 using 4-(bromomethyl)-1,2-difluorobenzene in the final step. ¹H NMR (400 MHz, CDCl₃) δ 7.92 (s, 1H), 7.76 (dd, 2H), 7.18 (dd, 2H), 7.00 (dd, 1H), 6.71 (dd, 2H), 5.92 (t, 1H), 4.37 (s, 3H), 3.68 (brd, 2H). LCMS: 386 (M+H)⁺.

### EXAMPLE 43

### 3-{[(4-Chlorophenyl)methyl]amino}-2-(4-fluorophenyl)-3,4-dihydropteridin-4-one

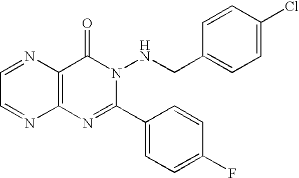

### Step 1: Methyl 3-(4-fluoro-N-(4-fluorobenzoyl)benzamido)pyrazine-2-carboxylate

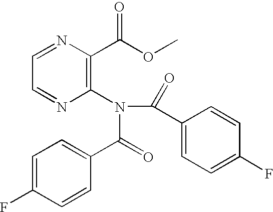

To a solution of methyl 3-aminopyrazine-2-carboxylate (15 g) in pyridine (360 mL) was added 4-fluorobenzoyl chloride (45 mL, 60 g) dropwise over 5 minutes. After 2 h at 70° C., the reaction mixture was concentrated. Saturated sodium bicarbonate solution was added (200 mL) to the crude residue. Water (2 L) was added and the solution was cooled to 4° C. for 16 h. The precipitate was collected by filtration to give methyl 3-(4-fluoro-N-(4-fluorobenzoyl)benzamido)pyrazine-2-carboxylate (35 g, 90%). ¹H NMR (400 MHz, DMSO-d₆) δ 8.75 (d, 1H), 8.66 (d, 1H), 7.84 (m, 4H), 7.31 (t, 4H), 3.79 (s, 3H). LCMS: 258 (M+H)⁺.

### Step 2: Methyl 3-(4-fluorobenzamido)pyrazine-2-carboxylate

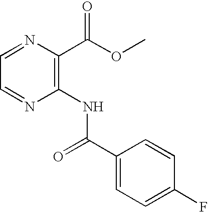

To a solution of methyl 3-(4-fluoro-N-(4-fluorobenzoyl)benzamido)pyrazine-2-carboxylate (35 g) in 5% IPA/Dioxane (400 mL), was added hydrazine (2.8 mL) while stirring. After 45 min at 85° C., cool down and concentrate to residue. Take up in 5% MeOH/DCM (200 mL), and filter. The filtrate is concentrated and purified by silica gel chromatography (25% to 100% ethyl acetate/hexanes) to give methyl 3-(4-fluorobenzamido)pyrazine-2-carboxylate (21 g, 88%). ¹H NMR (400 MHz, DMSO-d₆) δ 11.42 (s, 1H), 8.71 (d, 1H), 8.56 (d, 1H), 8.08 (m, 2H), 7.38 (t, 2H), 3.73 (s, 3H). LCMS: 276 (M+H)⁺.

### Step 3: 4-Fluoro-N-(3-(hydrazinecarbonyl)pyrazin-2-yl)benzamide

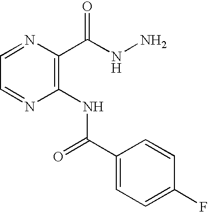

To a solution of methyl 3-(4-fluorobenzamido)pyrazine-2-carboxylate (20 g) in ethanol (400 mL), was added hydrazine (11.4 mL). After 16 h at RT, the reaction mixture is concentrated. Purification by silica gel chromatography (5% to 20% methanol/DCM) gives 4-fluoro-N-(3-(hydrazinecarbonyl)pyrazin-2-yl)benzamide (15 g, 75%) as a white solid. ¹H NMR (400 MHz, DMSO-d₆) δ 12.63 (s, 1H), 10.48 (s, 1H), 8.63 (d, 1H), 8.40 (d, 1H), 8.03 (m, 2H), 7.43 (t, 2H), 4.74 (s, 2H). LCMS: 276 (M+H)⁺.

### Step 4: 3-Amino-2-(4-fluorophenyl)pteridin-4(3H)-one

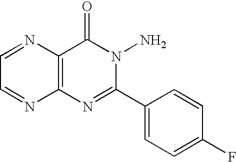

To a refluxing solution of methylbenzenesulfonic acid (380 mg) in toluene (3 L), was added 4-fluoro-N-(3-(hydrazinecarbonyl)pyrazin-2-yl)benzamide (10 g). After 3 days at reflux, the solution is cooled to 4° C. After 16 h at 4° C., the slurry is filtered to give 3-amino-2-(4-fluorophenyl)pteridin-4(3H)-one (6.3 g, 67%). ¹H NMR (400 MHz, CDCl₃) δ 9.03 (s, 1H), 8.89 (s, 1H), 8.07 (m, 2H), 7.28 (m, 2H), 5.19 (s, 2H). LCMS: 258 (M+H)⁺.

### Step 5: 3-{[(4-Chlorophenyl)methyl]amino}-2-(4-fluorophenyl)-3,4-dihydropteridin-4-one

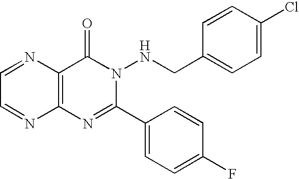

To a solution of 3-amino-2-(4-fluorophenyl)pteridin-4(3H)-one (2 g) in DMF (30 mL), was added sodium hydride (320 mg, 60%) and 1-(bromomethyl)-4-chlorobenzene (1.6 g). After 1 h at RT, methanol was added 1 mL). Purification by reverse phase HPLC (5% to 100% acetonitrile:water) gave 3-{[(4-chlorophenyl)methyl]amino}-2-(4-fluorophenyl)-3,4-dihydropteridin-4-one as a white solid. ¹H NMR (400 MHz, CD₃OD and CDCl3, TFA salt) δ 9.05 (s, 1H), 8.90 (s, 1H), 7.88 (dd, 2H), 7.20-7.10 (m, 4H), 6.89 (d, 2H), 3.91 (bs, 2H). LCMS: 382 (M+H)⁺.

### EXAMPLE 44

### 4-({[2-(4-Fluorophenyl)-4-oxo-3,4-dihydropteridin-3-yl]amino}methyl)benzonitrile

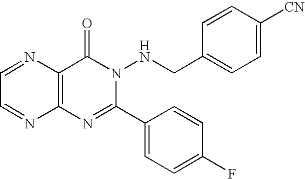

4-({[2-(4-Fluorophenyl)-4-oxo-3,4-dihydropteridin-3-yl]amino}methyl)benzonitrile was synthesized as described in EXAMPLE 43 using 4-(bromomethyl)benzonitrile in step 5. LCMS: 373 (M+H)⁺.

### EXAMPLE 45

### 3-{[(4-Chloro-3-fluorophenyl)methyl]amino}-2-(4-fluorophenyl)-3,4-dihydropteridin-4-one

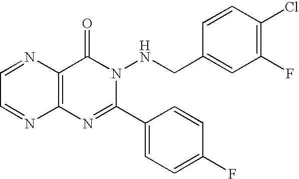

3-{[(4-Chloro-3-fluorophenyl)methyl]amino}-2-(4-fluorophenyl)-3,4-dihydropteridin-4-one was synthesized as described in EXAMPLE 43 using 4-(bromomethyl)-1-chloro-2-fluorobenzene in step 5. ¹H NMR (400 MHz, CD₃OD) δ 9.02 (s, 1H), 8.87 (s, 1H), 7.77 (dd, 2H), 7.24-7.15 (m, 3H), 6.78-6.70 (m, 2H), 3.97 (bs, 2H). LCMS: 400 (M+H)⁺.

### EXAMPLE 46

### 3-{[(4-Chloro-2-fluorophenyl)methyl]amino}-2-(4-fluorophenyl)-3,4-dihydropteridin-4-one

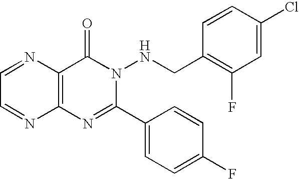

3-{[(4-Chloro-2-fluorophenyl)methyl]amino}-2-(4-fluorophenyl)-3,4-dihydropteridin-4-one was synthesized as described in EXAMPLE 43 using 1-(bromomethyl)-4-chloro-2-fluorobenzene in step 5. ¹H NMR (400 MHz, DMSO-d₆) δ 9.04 (s, 1H), 8.89 (s, 1H), 7.59 (m, 2H), 7.18 (t, 2H), 7.13-6.98 (m, 3H), 6.94 (t, 1H), 3.95 (bs, 2H). LCMS: 400 (M+H)⁺.

### EXAMPLE 47

### 3-{[(4-Chlorophenyl)methyl]amino}-2-(5-methylpyridin-3-yl)-3,4-dihydropteridin-4-one

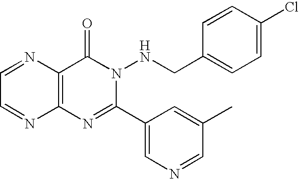

3-{[(4-Chlorophenyl)methyl]amino}-2-(5-methylpyridin-3-yl)-3,4-dihydropteridin-4-one was synthesized as described in EXAMPLE 43 using 5-methylnicotinoyl chloride in step 1. ¹H NMR (400 MHz, DMSO-d₆, HCl salt) δ 9.09 (s, 1H), 8.96 (s, 1H), 8.83 (s, 1H), 8.70 (s, 1H), 7.94 (s, 1H), 7.18 (bs, 1H), 7.13 (d, 2H), 6.83 (d, 2H) 3.95 (bs, 2H), 2.41 (s, 3H). LCMS: 379 (M+H)⁺.

### EXAMPLE 48

### 4-({[2-(5-Methylpyridin-3-yl)-4-oxo-3,4-dihydropteridin-3-yl]amino}methyl)benzonitrile

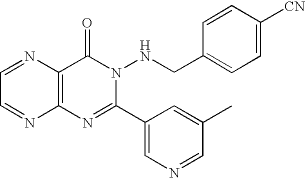

4-({[2-(5-Methylpyridin-3-yl)-4-oxo-3,4-dihydropteridin-3-yl]amino}methyl)benzonitrile was synthesized as described in EXAMPLE 43 using 5-methylnicotinoyl chloride in step 1, and 4-(bromomethyl)benzonitrile in step 5. ¹H NMR (400 MHz, DMSO-d₆, TFA salt) δ 9.07 (s, 1H), 8.92 (s, 1H), 8.62 (s, 1H), 8.51 (s, 1H), 7.65 (s, 1H), 7.53 (d, 2H), 7.20 (t, 1H), 7.01 (d, 2H), 4.06 (bs, 2H), 2.32 (s, 3H). LCMS: 370 (M+H)⁺.

### EXAMPLE 49

### 3-{[(4-Chlorophenyl)methyl]amino}-2-(5-chloropyridin-3-yl)-3,4-dihydropteridin-4-one

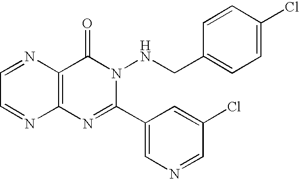

3-{[(4-Chlorophenyl)methyl]amino}-2-(5-chloropyridin-3-yl)-3,4-dihydropteridin-4-one was synthesized as described in EXAMPLE 43 using 5-chloronicotinoyl chloride (for a synthesis of 5-chloronicotinoyl chloride, see McElhinney, R. S., et. al.; J. Med. Chem.; 41; 26; 1998; 5625-5271) in step 1. ¹H NMR (400 MHz, CD₃OD, HCl salt) δ 9.07 (d, 1H), 8.93 (d, 1H), 8.92 (d, 1H), 8.88 (d, 1H), 8.33 (t, 1H), 7.13 (d, 2H), 6.88 (d, 2H), 4.04 (bs, 2H). LCMS: 399 (M+H)⁺.

### EXAMPLE 50

### 3-[(E)-[(4-Bromophenyl)methylidene]amino]-2-(4-fluorophenyl)-3,4-dihydropteridin-4-one

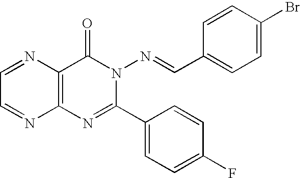

### Step 1: 3-Amino-2-(4-fluorophenyl)pteridin-4(3H)-one

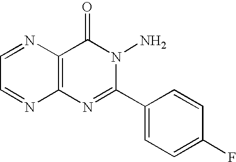

3-Amino-2-(4-fluorophenyl)pteridin-4(3H)-one was synthesized as described in EXAMPLE 43, steps 1 through 4. ¹H NMR (400 MHz, CDCl₃) δ 9.03 (s, 1H), 8.89 (s, 1H), 8.07 (m, 2H), 7.28 (m, 2H), 5.19 (s, 2H). LCMS: 258 (M+H)⁺.

### Step 2: 3-[(E)-[(4-Bromophenyl)methylidene]amino]-2-(4-fluorophenyl)-3,4-dihydropteridin-4-one

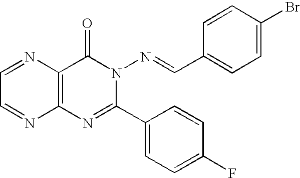

To 3-amino-2-(4-fluorophenyl)pteridin-4(3H)-one (50 mg) in DMF (2 mL), was added 4-bromobenzaldehyde (75 mg) and sodium triacetoxyborohydride (85 mg). After heating at 60° C. for 16 h, the reaction mixture is poured into ethyl acetate (20 mL), extracted three times with saturated sodium bicarbonate solution, and the organic layer is concentrated. The crude material is purified by silica gel chromatography (0% to 100% hexanes/ethyl acetate then 0% to 20% methanol/dichloromethane) to give 3-[(E)-[(4-bromophenyl)methylidene]amino]-2-(4-fluorophenyl)-3,4-dihydropteridin-4-one (42 mg, 51%) as a white solid. ¹H NMR (400 MHz, DMSO-d₆) δ 9.08 (s, 1H), 9.05 (s, 1H), 8.90 (s, 1H), 7.83-7.60 (m, 6H), 7.28 (t, 2H). LCMS: 424 (M+H)⁺.

### EXAMPLE 51

### 3-{[(4-Bromophenyl)methyl]amino}-2-(4-fluorophenyl)-3H,4H,5H,6H,7H,8H-pyrido[3,4-d]pyrimidin-4-one

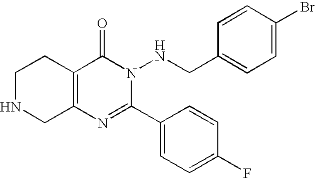

### Step 1: Methyl 1-benzyl-3-oxopiperidine-4-carboxylate

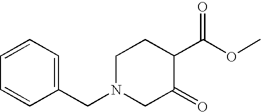

To a stirred mixture of 1-benzylpiperidin-3-one (72 g) and dimethyl carbonate (500 mL) was added NaH (38 g, 60%). After 20 minutes at reflux, water (800 mL) was added. The resulting solution was extracted with ethyl acetate (3×400 mL), dried over sodium sulfate and concentrated to give methyl 1-benzyl-3-oxopiperidine-4-carboxylate as a brown oil (93 g, 99%). LCMS: 248 (M+H)⁺.

### Step 2: Methyl 5-amino-1-benzyl-1,2,3,6-tetrahydropyridine-4-carboxylate

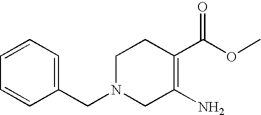

To a mixture of methyl 1-benzyl-3-oxopiperidine-4-carboxylate (10 g) and ammonium acetate (16 g), was added methanol (130 mL). After 2 h at RT, the mixture was concentrated. The residue was dissolved in DCM (150 mL). The mixture was washed with sodium carbonate solution (3×50 mL) and brine (2×50 mL), dried over sodium sulfate and concentrated. Purification by silica gel chromatography (1:3 ethyl acetate/petroleum ether) gave methyl 3-amino-1-benzyl-1,2,5,6-tetrahydropyridine-4-carboxylate (5.8 g, 58%) as a yellow solid. ¹H NMR (300 MHz, CDCl₃) δ 7.32 (m, 5H), 3.68 (s, 3H), 3.59 (s, 2H), 3.01 (s, 2H), 2.61 (t, 2H), 2.36 (t, 2H). LCMS: 247 (M+H)⁺.

### Step 3: 7-Benzyl-2-(4-fluorophenyl)-5,6,7,8-tetrahydro-4H-pyrido[3,4-d][1,3]oxazin-4-one

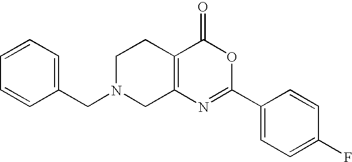

7-Benzyl-2-(4-fluorophenyl)-5,6,7,8-tetrahydro-4H-pyrido[3,4-d][1,3]oxazin-4-one was synthesized as described in EXAMPLE 31 steps 1 through 3 using methyl 5-amino-1-benzyl-1,2,3,6-tetrahydropyridine-4-carboxylate as starting material. LCMS: 337 (M+H)⁺.

### Step 4: tert-Butyl 2-(4-fluorophenyl)-4-oxo-5,6-dihydro-4H-pyrido[3,4-d][1,3]oxazine-7(8H)-carboxylate

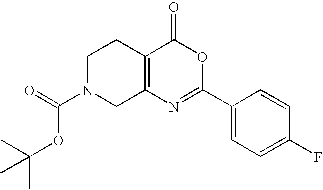

To a mixture of 7-benzyl-2-(4-fluorophenyl)-5,6,7,8-tetrahydropyrido[3,4-d][1,3]oxazin-4-one (7.4 g) and palladium on carbon (7 g, 10% Pd/C) in methanol (250 mL), was added d-tert-butyl dicarbonate (6.2 g). To the above mixture hydrogen gas was purged in. After 2 h at RT, the mixture was filtered, and the filtrate was concentrated to give tert-butyl 2-(4-fluorophenyl)-4-oxo-5,6-dihydro-4H-pyrido[3,4-d][1,3]oxazine-7(8H)-carboxylate (5.8 g, 76%) of as a yellow solid. ¹H NMR (300 MHz, CDCl₃) δ 8.26 (t, 2H), 7.21 (t, 2H), 4.39 (s, 2H), 3.68 (s, 2H), 2.64 (s, 2H), 1.53 (s, 9H). LCMS: 347 (M+H)⁺.

### Step 5: tert-Butyl 3-amino-2-(4-fluorophenyl)-4-oxo-3,4,5,6-tetrahydropyrido[3,4-d]pyrimidine-7(8H)-carboxylate

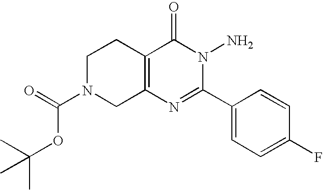

tert-Butyl 3-amino-2-(4-fluorophenyl)-4-oxo-3,4,5,6-tetrahydropyrido[3,4-d]pyrimidine-7(8H)-carboxylate was synthesized as described in EXAMPLE 1, step 3 using tert-butyl 2-(4-fluorophenyl)-4-oxo-5,6-dihydro-4H-pyrido[3,4-d][1,3]oxazine-7(8H)-carboxylate as starting material. ¹H NMR (300 MHz, CDCl₃) δ 7.87 (t, 2H), 7.31 (t, 2H), 5.75 (s, 2H), 4.29 (s, 2H), 3.57 (s, 2H), 2.50 (s, 2H), 1.43 (s, 9H). LCMS: 361 (M+H)⁺.

### Step 6: tert-Butyl 3-(4-bromobenzylamino)-2-(4-fluorophenyl)-4-oxo-3,4,5,6-tetrahydropyrido[3,4-d]pyrimidine-7(8H)-carboxylate

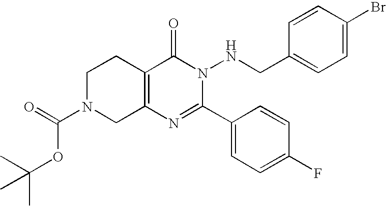

tert-Butyl 3-(4-bromobenzylamino)-2-(4-fluorophenyl)-4-oxo-3,4,5,6-tetrahydropyrido[3,4-d]pyrimidine-7(8H)-carboxylate was synthesized as described in EXAMPLE 1, step 4 using tert-butyl 3-amino-2-(4-fluorophenyl)-4-oxo-3,4,5,6-tetrahydropyrido[3,4-d]pyrimidine-7(8H)-carboxylate as starting material. LCMS: 529 (M+H)⁺.

### Step 7: 3-{[(4-Bromophenyl)methyl]amino}-2-(4-fluorophenyl)-3H,4H,5H,6H,7H,8H-pyrido[3,4-d]pyrimidin-4-one

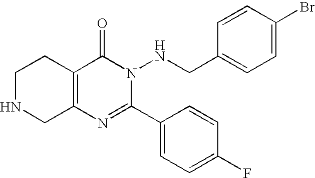

To a solution of tert-butyl 3-(4-bromobenzylamino)-2-(4-fluorophenyl)-4-oxo-3,4,5,6-tetrahydropyrido[3,4-d]pyrimidine-7(8H)-carboxylate (10 mg) in DCM (0.5 mL), was added trifluoroacetic acid (0.5 mL). After 2 h at RT, the reaction was diluted with ethyl acetate, dried over anhydrous sodium sulfate and concentrated to an oil. Purification by silica gel chromatography (100% ethyl acetate) gave 3-{[(4-bromophenyl)methyl]amino}-2-(4-fluorophenyl)-3H,4H,5H,6H,7H,8H-pyrido[3,4-d]pyrimidin-4-one (7 mg, 84% yield). ¹H NMR (400 MHz, CDCl₃) δ 7.79 (dd, 2H), 7.37 (d, 2H), 7.12 (dd, 2H), 6.81 (d, 2H), 4.12 (brt, 2H), 3.71 (brd, 2H), 3.44 (brt, 2H), 2.99 (brt, 2H), 2.52 (brm, 1H). LCMS: 430 (M+H)⁺.

The following compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36. All SMILES strings used herein, as well as many IUPAC names, were generated using CambridgeSoft's ChemDraw 10.0.
[N]═C(N═C(C1=CC═C(O)C(C(F)(F)F)═C1)N(NCC2=CC═C(Cl)C═C2)C3=O)C3=[N]
O═C4N(NCC5=CC═C(Cl)C═C5)C(C6=CC═C(O)C(C#N)═C6)=NC7=NC═CN═C74
O═C8N(NCC9=CC═C(Cl)C═C9)C(C%10=CC═C(O)C(Cl)═C%10)=NC%11=NC═CN═C%118
O═C%12N(NCC%13=CC═C(Cl)C═C%13)C(C%14=CC═C(O)C(OC(F)(F)F)═C%14)=NC%15=NC═CN═C%15%12
O═C%16N(NCC%17=CC═C(Cl)C═C%17)C(C%18=CC═C(O)C(OC%19=CN═CC═C%19)=C%18)=NC%20=NC═CN═C%20%16
O═C%21N(NCC%22=CC═C(Cl)C═C%22)C(C%23=CC═C(N)C(C(F)(F)F)═C%23)=NC%24=NC═CN═C%24%21
O═C%25N(NCC%26=CC═C(Cl)C═C%26)C(C%27=CC═C(N)C(C#N)═C%27)=NC%28=NC═CN═C%28%25
O═C%29N(NCC%30=CC═C(Cl)C═C%30)C(C%31=CC═C(N)C(Cl)═C%31)=NC%32=NC═CN═C%32%29
O═C%33N(NCC%34=CC═C(Cl)C═C%34)C(C%35=CC═C(N)C(OC(F)(F)F)═C%35)=NC%36=NC═CN═C%36%33
O═C%37N(NCC%38=CC═C(Cl)C═C%38)C(C%39=CC═C(N)C(OC%40=CN═CC═C%40)=C%39)=NC%41=NC═CN═C%41%37
O═C%42N(NCC%43=CC═C(Cl)C═C%43)C(C%44=CC(C%45=CC═CC═C%45)=C(N)C═C%44)=NC%46=NC═CN═C%46%42
O═C%47N(NCC%48=CC═C(Cl)C═C%48)C(C%49=CC(C%50=CC═CC═C%50)=C(O)C═C%49)=NC%51=NC═CN═C%51%47
O═C%52N(NCC%53=CC═C(Cl)C═C%53)C(C%54=CC═C(N)C(N(C)CC%55=CN═CC═C%55)=C%54)=NC%56=NC═CN═C%56%52
O═C%57N(NCC%58=CC═C(Cl)C═C%58)C(C%59=CC═C(N)C(OCC(C)(C)C)═C%59)=NC%60=NC═CN═C%60%57
O═C1N(NCC2=CC═C(Cl)C═C2)C(C3=CC═C(NC(C)═O)C(C)═C3)=NC4=NC═CN═C41
O═C5N(NCC6=CC═C(Cl)C═C6)C(C7=CC═C(NS(C)(═O)═O)C(C)═C7)=NC8=NC═CN═C85
O═C9N(NCC%10=CC═C(Cl)C═C%10)C(C%11=CC═C(NS(C%12=CC═CC═C%12)(═O)═O)C(C)═C%11)=NC%13=NC═CN═C%139
O═C%14N(NCC%15=CC═C(Cl)C═C%15)C(C%16=CC═[N+]([O—])C(C)═C%16)=NC%17=NC═CN═C%17%14
O═C%18N(NCC%19=CC═C(Cl)C═C%19)C(C%20=CC(CO)═CC═C%20)=NC%21=NC═CN═C%21%18
O═C%22N(NCC%23=CC═C(Cl)C═C%23)C(C%24=CC(C(N)═O)═CC═C%24)=NC%25=NC═CN═C%25%22
O═C%26N(NCC%27=CC═C(Cl)C═C%27)C(C%28=CC(C(C)(C)O)═CC═C%28)=NC%29=NC═CN═C%29%26
O═C%30N(NCC%31=CC═C(Cl)C═C%31)C(C%32=CC═C(C(O)(C)C)C═C%32)=NC%33=NC═CN═C%33%30
O═C%34N(NCC%35=CC═C(Cl)C═C%35)C(C%36=CC(C%37=CC═CC═N%37)=CC═C%36)=NC%38=NC═CN═C%38%34
O═C%39N(NCC%40=CC═C(Cl)C═C%40)C(C%41=CC(C%42=CC═CC═C%42CO)═CC═C%41)=NC%43=NC═CN═C%43%39
O═C%44N(NCC%45=CC═C(Cl)C═C%45)C(C%46=CC(OC%47=CC═C[N+]([O—])═C%47)=CC═C%46)=NC%48=NC═CN═C%48%44
O═C%49N(NCC%50=CC═C(Cl)C═C%50)C(C%51=CC(C%52=CC═NO%52)=CC═C%51)=NC%53=NC═CN═C%53%49
O═C%54N(NCC%55=CC═C(Cl)C═C%55)C(C%56=CC(C%57=NC═CN%57C)═CC═C%56)=NC%58=NC═CN═C%58%54
O═C%59N(NCC%60=CC═C(Cl)C═C%60)C(C%61=CC(OC%62=CN═CO%62)=CC═C%61)=NC%63=NC═CN═C%63%59
O═C%64N(NCC%65=CC═C(Cl)C═C%65)C(C%66=CC═C(C(N)═O)C═C%66)=NC%67=NC═CN═C%67%64
O═C%68N(NCC%69=CC═C(Cl)C═C%69)C(C%70=CC═C(C(N)═O)C(C#N)═C%70)=NC%71=NC═CN═C%71%68
O═C%72N(NCC%73=CC═C(Cl)C═C%73)C(C%74=CC═C(C(N)═O)C(C)═C%74)=NC%75=NC═CN═C%75%72
O═C%76N(NCC%77=CC═C(Cl)C═C%77)C(C%78=CC(C(C)(C)O)═CN═C%78)=NC%79=NC═CN═C%79%76
O═C1N(NCC2=CC═C(Cl)C═C2)C(C3=CC═C(O)C(OC)═C3)=NC4=NC═CN═C41
O═C5N(NCC6=CC═C(Cl)C═C6)C(C7=CC═C(O)C(C)═C7)=NC8=NC═CN═C85
O═C9N(NCC%10=CC═C(Cl)C═C%10)C(C%11=CN═CC(OC)═C%11)=NC%12=NC═CN═C%129
O═C%13N(NCC%14=CC═C(Cl)C═C%14)C(C%15=CN═CC(Cl)═C%15)=NC%16=NC═CN═C%16%13
O═C%17N(NCC%18=CC═C(Cl)C═C%18)C(C%19=CN═CC(C)═C%19)=NC%20=NC═CN═C%20%17
O═C%21N(NCC%22=CC═C(Cl)C═C%22)C(C%23=CN═CS%23)=NC%24=NC═CN═C%24%21
O═C%25N(NCC%26=CC═C(Cl)C═C%26)C(C%27=CC═CC(OC)═C%27)=NC%28=NC═CN═C%28%25
O═C%29N(NCC%30=CC═C(Cl)C═C%30)C(C%31CCCCC%31)=NC%32=NC═CN═C%32%29
O═C%33N(NCC%34=CC═C(Cl)C═C%34)C(CC%35=CC═CN═C%35)=NC%36=NC═CN═C%36%33
O═C%37N(NCC%38=CC═C(Cl)C═C%38)C(OC%39=CC(Cl)═CC(F)═C%39)=NC%40=NC═CN═C%40%37
O═C%41N(NCC%42=CC═C(Cl)C═C%42)C(N%43CCN(C)CC%43)=NC%44=NC═CN═C%44%41
O═C%45N(NCC%46=CC═C(Cl)C═C%46)C(C%47=CN═C(C)S%47)=NC%48=NC═CN═C%48%45
O═C%49N(NCC%50=CC═C(Cl)C═C%50)C(C%51=CN═C(O)S%51)=NC%52=NC═CN═C%52%49
O═C%53N(NCC%54=CC═C(Cl)C═C%54)C(C%55=C(C)N═CS%55)=NC%56=NC═CN═C%56%53
O═C%57N(NCC%58=CC═C(Cl)C═C%58)C(C%59=C(O)N═CS%59)=NC%60=NC═CN═C%60%57
O═C%61N(NCC%62=CC═C(Cl)C═C%62)C(C%63=CN═C(C)N%63)=NC%64=NC═CN═C%64%61
O═C%65N(NCC%66=CC═C(Cl)C═C%66)C(C%67=NN═C(O)S%67)=NC%68=NC═CN═C%68%65
O═C%69N(NCC%70=CC═C(Cl)C═C%70)C(C%71=C(C)N═CO%71)=NC%72=NC═CN═C%72%69
O═C%73N(NCC%74=CC═C(Cl)C═C%74)C(C%75=C(O)N═NS%75)=NC%76=NC═CN═C%76%73
O═C%77N(NCC%78=CC═C(Cl)C═C%78)C(C%79=NN═C(N)N%79C)═NC%80=NC═CN═C%80%77
O═C1N(NCC2=CC═C(Cl)C═C2)C(C3=CC═C(O)C(OC)═C3)=NC4=NC(C)═CN═C41
O═C5N(NCC6=CC═C(Cl)C═C6)C(C7=CN═CC(OC)═C7)=NC8=NC═C(C)N═C85
O═C9N(NCC%10=CC═C(Cl)C═C%10)C(C%11=CN═CC(Cl)═C%11)=NC%12=NC(C%13=CC═CC═C%13)=CN═C%129
O═C%14N(NCC%15=CC═C(Cl)C═C%15)C(C%16=CN═CC(C)═C%16)=NC%17=NC(OC)═CN═C%17%14
O═C%18N(NCC%19=CC═C(Cl)C═C%19)C(C%20=CN═CS%20)=NC%21=NC(C(F)(F)F)═CN═C%21%18
O═C%22N(NCC%23=CC═C(Cl)C═C%23)C(C%24=CC═CC(OC)═C%24)=NC%25=NC═C(C%26=CC═CC═C%26)N═C%25%22
O═C%27N(NCC%28=CC═C(C(F)(F)F)C(F)═C%28)C(C%29=CC═C(O)C(OC)═C%29)=NC%30=NC═CN═C%30%27
O═C%31N(NCC%32=CC═C(Cl)C═C%32)C(C%33=CC═C(N)C(C)═C%33)=NC%34=NC═CN═C%34%31
O═C%35N(NCC%36=CC═C(Cl)C═N%36)C(C%37=CN═CC(OC)═C%37)=NC%38=NC═CN═C%38%35
O═C%39N(NCC%40=CC═C(F)C═C%40F)C(C%41=CN═CC(C)═C%41)=NC%42=NC═CN═C%42%39
O═C%43N(NCC%44=NN(C)C═C%44)C(C%45=CN═CS%45)=NC%46=NC═CN═C%46%43
O═C%47N(NCC%48=CC═C(Cl)C═C%48F)C(C%49=CC═CC(OC)═C%49)=NC%50=NC═CN═C%50%47
O═C%51N(NCC%52=CC═C(C)S%52)C(C%53=CN═CC(Cl)═C%53)=NC%54=NC═CN═C%54%51
O═C%55N(N(C)CC%56=CC═C(Cl)C═C%56)C(C(\C═C(C)/C)═C)═NC%57=NC═CN═C%57%55
O═C%58N(/N═C/C%59=CC═C(Cl)C═C%59)C(C(\C═C(OC)/C)═C)═NC%60=NC═CN═C%60%58
O═C%61N(NC(C)C%62=CC═C(Cl)C═C%62)C(C/C═C/Cl)═NC%63=NC═CN═C%63%61
O═C%64N(NCCC%65=CC═C(Cl)C═C%65)C(C/C═C/C)═NC%66=NC═CN═C%66%64
O═C%67N(NCC%68=CC═C(Cl)C═C%68)C(C%69=CN═CC(OC%70CC%70)=C%69)=NC%71=NC═C(OC)N═C%71%67
O═C%72N(NCC%73=CC═C(Cl)C═C%73)C(C%74=CN═CS%74)=NC%75=NC═C(C(F)F)N═C%75%72
O═C%76N(NCC%77=CC═C(C(F)F)C(F)═C%77)C(C%78=CC═C(O)C(C)═C%78)=NC%79=NC═CN═C%79%76
O═C%80N(NCC%81=CC═C(C(F)(F)F)C═C%81)C(C%82=CC═C(N)C(C)═C%82)=NC%83=NC═CN═C%83%80
O═C%84N(N(C%85=CC═C(Cl)C═C%85)C)C(C/C═C/C)═NC%86=NC═CN═C%86%84
O═C%87N(NCC%88CCCCC%88)C(C%89=CC═CC(C#N)═C%89)=NC%90=NC═CN═C%90%87
O═C1N(NCC2=CC═C(Cl)C═C2)C(C3=CN═CS3)=NC4=NC═C(C)N═C41
O═C5N(NCC6=CC═C(Cl)C═C6)C(C7=CN═CN7)=NC8=NC(C)═C(C)N═C85
O═C9N(NCC%10=CC═C(C#N)C═C%10)C(C%11=CC═C(O)C(C(F)(F)F)═C%11)=NC%12=NC═CN═C%129
O═C%13N(NCC%14=CC═C(C(C)(C)C)C═C%14)C(C%15=CC═C(O)C(C#N)═C%15)=NC%16=NC═CN═C%16%13
O═C%17N(NCC%18=CC═C(OC(F)(F)F)C═C%18)C(C%19=CC═C(O)C(Cl)═C%19)=NC%20=NC═CN═C%20%17
O═C%21N(NCC%22=CC═C(Cl)C(Cl)═C%22)C(C%23=CC═C(O)C(OC(F)(F)F)═C%23)=NC%24=NC═CN═C%24%21
O═C%25N(NCC%26=CC(C)═CC(F)═C%26)C(C%27=CC═C(O)C(OC%28=CN═CC═C%28)=C%27)=NC═CN═C%29%25
O═C%30N(NCC%31=C(Cl)C═CC(C#N)═C%31)C(C%32=CC(C%33=CC═CC═C%33)=C(O)C═C%32)=NC%34=NC═CN═C%34%30
O═C%35N(NCC%36=CC═C(C#N)C═C%36)C(C%37=CC═C(O)C(C(F)(F)F)═C%37)=NC%38=NC(C)═CN═C%38%35
O═C%39N(NCC%40=CC═C(C(C)(C)C)C═C%40)C(C%41=CC═C(O)C(C#N)═C%41)=NC%42=NC═CN═C%42%39
O═C%43N(NCC%44=CC═C(OC(F)(F)F)C═C%44)C(C%45=CC═C(O)C(Cl)═C%45)=NC%46=NC═CN═C%46%43
O═C%47N(NCC%48=CC═C(Cl)C(Cl)═C%48)C(C%49=CC═C(O)C(OC(F)(F)F)═C%49)=NC%50=NC═CN═C%50$47
O═C%51N(NCC%52=CC(Cl)═C(C)C(F)═C%52)C(C%53=CC═C(O)C(OC%54=CN═CC═C%54)=C%53)=NC%55=NC═C%55%51
O═C%56N(NCC%57=C(Cl)C═C(Br)C(C#N)═C%57)C(C%58=CC(C%59=CC═CC═C%59)=C(N)C═C%58)=NC%60=NC═CN═C%60%56
O═C%61N(NCC%62=C(F)C═C(Br)C═C%62F)C(C%63=CC(C%64=CC═CN═C%64)=C(N)C═C%63)=NC%65=NC═CN═C%65%61
O═C1N(NCC2=CC═C(Cl)C═C2)C(C3=CC═C(O)C(C(F)(F)F)═C3)=NC4=NC═CC═C41
O═C5N(NCC6=CC═C(Cl)C═C6)C(C7=CC═C(O)C(C#N)═C7)=NC8=NC═NC═C85
O═C9N(NCC%10=CC═C(Cl)C═C%10)C(C%11=CC═C(O)C(Cl)═C%11)=NC%12=CN═CC═C%129
O═C%13N(NCC%14=CC═C(Cl)C═C%14)C(C%15=CC═C(O)C(OC(F)(F)F)═C%15)=NC%16=NC═CC═C%16%13
O═C%17N(NCC%18=CC═C(Cl)C═C%18)C(C%19=CC═C(O)C(OC%20=CN═CC═C%20)=C%19)=NC%21=CC═CN═C%21%17
O═C%22N(NCC%23=CC═C(Cl)C═C%23)C(C%24=CC═C(N)C(C(F)(F)F)═C%24)=NC%25=CC═NC═C%25%22
O═C%26N(NCC%27=CC═C(Cl)C═C%27)C(C%28=CC═C(N)C(C#N)═C%28)=NC%29=NC═CC═C%29%26
O═C%30N(NCC%31=CC═C(Cl)C═C%31)C(C%32=CC═C(N)C(Cl)═C%32)=NC%33=NC═CC═C%33%30
O═C%34N(NCC%35=CC═C(Cl)C═C%35)C(C%36=CC═C(N)C(OC(F)(F)F)═C%36)=NC%37=CN═CC═C%37%34
O═C%38N(NCC%39=CC═C(Cl)C═C%39)C(C%40=CC(C%41=CC═CC═C%41)=C(N)C═C%40)=NC%42=NC═CC═C%42%38
O═C%43N(NCC%44=CC═C(Cl)C═C%44)C(C%45=CC(C%46=CC═CC═C%46)=C(O)C═C%45)=NC%47=NC═CC═C%47%43
O═C%48N(NCC%49=CC═C(Cl)C═C%49)C(C%50=CC═C(NC(C)═O)C(C)═C%50)=NC%51=CN═CN═C%51%48
O═C%52N(NCC%53=CC═C(Cl)C═C%53)C(C%54=CC═C(NS(C)(═O)═O)C(C)═C%54)=NC%55=CC═CN═C%55%52
O═C%56N(NCC%57=CC═C(Cl)C═C%57)C(C%58=CC═C(NS(C%59=CC═CC═C%59)(═O)═O)C(C)═C%58)=NC%60=NC═CC═C%60%56
O═C%61N(NCC%62=CC═C(Cl)C═C%62)C(C%63=CC═[N+]([O—])C(C)═C%63)=NC%64=CC═NC═C%64%61
O═C%65N(NCC%66=CC═C(Cl)C═C%66)C(C%67=CC(CO)═CC═C%67)=NC%68=CC═CN═C%68%65
O═C%69N(NCC%70=CC═C(Cl)C═C%70)C(C%71=CC(C(N)═O)═CC═C%71)=NC%72=CN═CC═C%72%69
O═C%73N(NCC%74=CC═C(Cl)C═C%74)C(C%75=CC(C(C)(C)O)═CC═C%75)=NC%76=NC═CC═C%76%73
O═C%77N(NCC%78=CC═C(Cl)C═C%78)C(C%79=CC═C(C(O)(C)C)C═C%79)=NC%80=NC═CC═C%80%77
O═C%81N(NCC%82=CC═C(Cl)C═C%82)C(C%83=CC(C%84=CC═CC═N%84)=CC═C%83)=NC%85=NC═CC═C%85%81
O═C%86N(NCC%87=CC═C(Cl)C═C%87)C(C%88=CC(C%89=CC═CC═C%89CO)═CC═C%88)=NC%90=CC═CN═C%90%86
O═C%91N(NCC%92=CC═C(Cl)C═C%92)C(C%93=CC(OC%94=CC═C[N+]([O—])═C%94)=CC═C%93)=NC%95=CN═CC═C%95%91
O═C%96N(NCC%97=CC═C(Cl)C═C%97)C(C%98=CC(C%99=CC═NO%99)=CC═C%98)=NC%100=NC═CC═C%100%96
O═C%101N(NCC%102=CC═C(Cl)C═C%102)C(C%103=CC(C%104=NC═CN%104C)═CC═C%103)=NC%105=NC═CC═C%105%101
O═C%106N(NCC%107=CC═C(Cl)C═C%107)C(C%108=CC(OC%109=CN═CO%109)=CC═C%108)=NC%110=CN═CC═C%110%106
O═C%111N(NCC%112=CC═C(Cl)C═C%112)C(C%113=CC═C(N)C(N(C)CC%114=CN═CC═C%114)=C%113)=NC%115=NC═CC═C%115%111
O═C%116N(NCC%117=CC═C(Cl)C═C%117)C(C%118=CC═C(C(N)═O)C═C%118)=NC%119=CN═CC═C%119%116
O═C%120N(NCC%121=CC═C(Cl)C═C%121)C(C%122=CC═C(C(N)═O)C(C#N)═C%122)=NC%123=NC═CC═C%123%120
O═C%124N(NCC%125=CC═C(Cl)C═C%125)C(C%126=CC═C(C(N)═O)C(C)═C%126)=NC%127=NC═CC═C%127%124
O═C%128N(NCC%129=CC═C(Cl)C═C%129)C(C%130=CC(C(C)(C)O)═CN═C%130)=NC%131=CN═CC═C%131%128
O═C%132N(NCC%133=CC═C(Cl)C═C%133)C(C%134=CC═C(N)C(OCC(C)(C)C)═C%134)=NC%135=NC═CC═C%135%132

The activity of the compounds in Examples 1-51 as TGR5 modulators is illustrated in the following assay. The other compounds listed above, which have not yet been made and/or tested, are predicted to have activity in this assay as well.

### Biological Activity Assay

cAMP Production Assay:

HEK293 cells stably expressing TGR5 (HEK293-TGR5) were established by stably transfecting HEK-293 cells with an expression vector (pcDNA 3.1, Invetrogen) inserted with human TGR5 cDNA using Fugene6 (Roche, Indianapolis, Ind.) according to conventional methods. Cells were grown in DMEM (invitrogen, Carlsbad, Calif.) supplemented with 10% FBS, 1% penicillin/streptomycin under geneticin selection. The presence of TGR5 transcripts in these cells was confirmed using branched DNA (bDNA, Genospectra, Inc., Fremont Calif.) following the manufacturer's protocol and using specific probes for human TGR5. cAMP production assay was performed in high throughput 1536 well format using LANCE cAMP detection kit (Perkin Elmer Inc., Boston, Mass.) according to the manufacturer's protocol. Briefly, HEK293-TGR5 cells were harvested using non-enzymatic cell dissociation buffer (Invitrogen, Carlsbad, Calif.) and suspended in DMEM supplemented with 0.1% FBS at a density of 800,000 cells/ml. Alexa antibody was added to the cell suspension, and 4 ul of the mixture was dispensed in white opaque tissue culture treated Greiner 1536 well plates (USA Scientific, Inc., Ocala, Fla.). After an overnight incubation at 37 C in an atmosphere of 10% CO2 and 95% humidity, 1 ul of 5 mM IBMX (Sigma, St. Louis, Mo.) solution in DMEM was dispensed for a final concentration of 1 mM. Cells were then stimulated with test compounds for 30 minutes, after which time 5 ul of detection reagent was added and incubated for 1-7 hrs at room temperature. TR-FRET signal was detected using the Viewlux (Perkin Elmer Inc., Boston Mass.). EC₅₀ values were determined using Graph Pad Prizm analysis (GraphPad Software, Inc). The EC₅₀ values for a wide range of bile acids generated from this assay were in agreement with the values published in the scientific literature. None of the compounds induced cAMP in HEK-293 cells that were transfected with an empty vector alone, confirming a TGR5 mechanism of action for cAMP production. The symbol (+) denotes an EC₅₀ value of ≦10 μM while the symbol (−) denotes an EC₅₀ value of >10 μM (see Table 1).

TABLE 1
Biological Activity

| | cAMP Production in |
|---|---|
| | 293-TGR5 Cells; EC50: |
| Example | (+): ≦10 μM; |
| No. | (−): >10 μM |
| 1 | + |
| 2 | + |
| 3 | + |
| 4 | + |
| 5 | + |
| 6 | + |
| 7 | + |
| 8 | + |
| 9 | + |
| 10 | + |
| 11 | + |
| 12 | + |
| 13 | + |
| 14 | + |
| 15 | + |
| 16 | + |
| 17 | + |
| 18 | + |
| 19 | + |
| 20 | + |
| 21 | + |
| 22 | + |
| 23 | + |
| 24 | + |
| 25 | + |
| 26 | + |
| 27 | + |
| 28 | + |
| 29 | + |
| 30 | + |
| 31 | + |
| 32 | + |
| 33 | + |
| 34 | + |
| 35 | + |
| 36 | + |
| 37 | + |
| 38 | + |
| 39 | + |
| 40 | + |
| 41 | + |
| 42 | − |
| 43 | + |
| 44 | + |
| 45 | + |
| 46 | + |
| 47 | + |
| 48 | + |
| 49 | + |
| 50 | − |
| 51 | − |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula II: 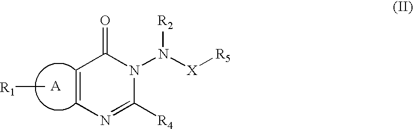
or a salt, ester, or prodrug thereof, wherein:
A is a 5 or 6-membered monocyclic heterocycloalkyl or heteroaryl;
X is selected from the group consisting of C(R₆)(R₇) and C(R₆)(R₇)C(R₈)(R₉).
R₁ is selected from the group consisting of hydrogen, halogen, amino, cyano, nitro, hydroxy, alkoxy, alkyl, acyl, alkenyl, alkynyl, heteroalkyl, carboxyl, alkylthio, alkylsulfonyl, sulfonamido, aryl, arylalkyl, cycloalkyl, cycloalkylalkyl, haloalkyl, perhaloalkyl, perhaloalkoxy, heteroaryl, heteroarylalkyl, heterocycloalkyl, and heterocycloalkylalkyl, any of which may be optionally substituted;
R₂ is selected from the group consisting of hydrogen, alkyl, acyl, alkenyl, alkynyl, heteroalkyl, aryl, arylalkyl, cycloalkyl, cycloalkylalkyl, haloalkyl, perhaloalkyl, heteroaryl, heteroarylalkyl, heterocycloalkyl, and heterocycloalkylalkyl, any of which may be optionally substituted, or R₂ and R₆, taken together, form a bond;
R₄ and R₅ are independently selected from the group consisting of aryl, cycloalkyl, heteroaryl, and heterocycloalkyl, any of which may be optionally substituted; and
R₆, R₇, R₈, and R₉ are independently selected from the group consisting of hydrogen and optionally substituted lower alkyl, or R₆ and R₇ or R₈ and R₉, taken together, are oxo or saturated C₃-C₆ cycloalkyl.

2. The compound as recited in claim 1, wherein:
X is selected from the group consisting of CH₂ and CH₂CH₂; and
R₂ is selected from the group consisting of hydrogen and lower alkyl.

3. The compound as recited in claim 2, wherein:
A is a 5 or 6-membered monocyclic heteroaryl.

4. The compound as recited in claim 3, wherein:
R₄ and R₅ are independently selected from the group consisting of phenyl, napthyl, monocyclic heteroaryl, and bicyclic heteroaryl, any of which may be optionally substituted with one or more substituents selected from the group consisting of hydrogen, halogen, hydroxyl, nitro, cyano, amino, lower alkyl, lower alkoxy, and lower perhaloalkyl.

5. The compound as recited in claim 4, having a structural formula selected from the group consisting of Formula III and Formula IV: 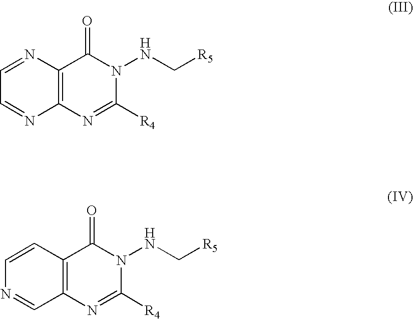
or a salt, ester, or prodrug thereof, wherein R₄ and R₅ are independently selected from the group consisting of phenyl, napthyl, monocyclic heteroaryl, and bicyclic heteroaryl, any of which may be optionally substituted with one or more substituents selected from the group consisting of hydrogen, halogen, hydroxyl, nitro, cyano, amino, lower alkyl, lower alkoxy, and lower perhaloalkyl.

6. The compound as recited in claim 5, wherein R₄ and R₅ are each phenyl, which may be optionally substituted with one or more substituents selected from the group consisting of hydrogen, halogen, hydroxyl, nitro, cyano, amino, lower alkyl, lower alkoxy, and lower perhaloalkyl.

7. The compound as recited in claim 6, wherein:
R₅ is 4-halophenyl.

8. The compound as recited in claim 7, wherein:
R₄ is selected from the group consisting of 5-chloro-pyridin-3-yl, 5-methyl-pyridin-3-yl, 4-fluorophenyl, 4-amino-3-methylphenyl, 4-hydroxy-3-methylphenyl, and 4-hydroxy-3-methoxyphenyl.

9. The compound as recited in claim 1 selected from the group consisting of Examples 1 to 51.

10. A pharmaceutical composition comprising a compound as recited in claim 1 together with a pharmaceutically acceptable carrier.

11. The pharmaceutical composition as recited in claim 10, useful for the treatment or prevention of a TGR5-mediated disease.

12. A method of treatment of a TGR5-mediated disease comprising the administration of a therapeutically effective amount of a compound as recited in claim 1 to a patient in need thereof.

13. The method as recited in claim 12, wherein said disease is a metabolic disease.

14. The method as recited in claim 13, wherein said disease is selected from the group consisting of inadequate glucose tolerance, insulin resistance, type I diabetes, and type II diabetes.

15. The method as recited in claim 12, further comprising the administration of another therapeutic agent.

16. The method as recited in claim 15, wherein said agent is selected from the group consisting of insulin, metformin, Glipizide, glyburide, Amaryl, gliclazide, meglitinides, nateglinide, repaglinide, pramlintide, PTP-112, SB-517955, SB-4195052, SB-216763, NN-57-05441, NN-57-05445, GW-0791, AGN-¹⁹4²⁰4, T-1095, BAY R3401, acarbose, miglitol, voglibose, Exendin-4, DPP728, LAF237, vildagliptin, BMS477118, PT-100, GSK-823093, PSN-9301, T-6666, SYR-322, SYR-619, Liraglutide, CJC-1134-PC, naliglutide, MK-0431, saxagliptin, GSK23A, pioglitazone, rosiglitazone, AVE2268, GW869682, GSK189075, APD668, PSN-119-1, PSN-821, rosuvastatin, atrovastatin, simvastatin, lovastatin, pravastatin, fluvastatin, cerivastatin, rosuvastatin, pitavastatin, fenofibrate, benzafibrate, clofibrate, gemfibrozil, Ezetimibe, eflucimibe, CP-529414, CETi-1, JTT-705, cholestyramine, colestipol, niacin, implitapide, (R)-1-{4-[5-methyl-2-(4-trifluoromethyl-phenyl)-oxazol-4-ylmethoxy]-benzenesulfonyl}2,3-dihydro-1H-indole-2-carboxylic acid, and GI-262570.

17. The method as recited in claim 13 wherein said disease is associated with perturbed bile acid metabolism.

18. The method as recited in claim 17 further comprising the administration of another therapeutic agent.

19. The method as recited in claim 12 wherein said disease is an inflammatory disease.

20. The method as recited in claim 19 wherein said disease is selected from the group consisting of rheumatoid arthritis, ulcerative colitis, and inflammatory bowel disease.

21. The method as recited in claim 20 further comprising the administration of another therapeutic agent.

22. The method as recited in claim 21, wherein said agent is selected from the group consisting of betamethasone dipropionate, betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocaine, EMLA Cream, guaifenesin, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin, clonidine, codeine, loperamide, tramadol, morphine, fentanyl, oxycodone, hydrocodone, levorphanol, butorphanol, menthol, oil of wintergreen, camphor, eucalyptus oil, turpentine oil, acetaminophen, infliximab, etanerecept, infliximab, and capsaicin.

23. The method as recited in claim 12 wherein said disease is obesity.

24. The method as recited in claim 23 wherein said method achieves an effect selected from the group consisting of decreasing body weight and controlling weight gain.

25. The method as recited in claim 23 further comprising the administration of another therapeutic agent.

26. The method as recited in claim 25, wherein said agent is selected from the group consisting of sibutramine, bromocriptine, Orlistat, rimonabant, Axokine, and bupropion.

27. A method for achieving an effect in a patient comprising the administration of a therapeutically effective amount of a compound as recited in claim 1 to a patient, wherein the effect is selected from the group consisting of improving glucose tolerance, decreasing insulin resistance, decreasing body weight, controlling weight gain, modulation of type I diabetes, modulation of type II diabetes, modulation of perturbed bile acid metabolism, modulation of rheumatoid arthritis, modulation of ulcerative colitis, and modulation of inflammatory bowel disease.

28. A method of modulating TGR5 comprising contacting TGR5 with a compound as recited in claim 1.

29. A compound as recited in claim 1 for use as a medicament.

30. A compound as recited in claim 1 for use in the manufacture of a medicament for the prevention or treatment of a disease or condition ameliorated by the modulation of TGR5.
